(19) United States
(12) Patent Application Publication
(10) Pub. No.: US 2021/0301269 A1
(43) Pub. Date: Sep. 30, 2021

(54) RECOMBINANT CRISPR-CAS9 NUCLEASES WITH ALTERED PAM SPECIFICITY

(71) Applicants: New York Genome Center, Inc., New York, NY (US); New York University, New York, NY (US)

(72) Inventors: Neville E. Sanjana, New York, NY (US); Zharko Daniloski, New York, NY (US); Mateusz Legut, Jersey City, NJ (US)

(21) Appl. No.: 17/154,826

(22) Filed: Jan. 21, 2021

Related U.S. Application Data
(60) Provisional application No. 62/964,483, filed on Jan. 22, 2020.

(51) Int. Cl. C12N 9/22; C07K 14/47; C12N 7/00; C12N 15/11; C12N 15/10

(52) U.S. Cl. CPC C12N 9/22; C07K 14/4702; C12N 7/00; C07K 2319/00; C12N 15/1082; C12N 2310/20; C12N 15/111

(57) ABSTRACT

Provided herein is a recombinant or engineered Cas9 protein. The Cas9 protein has an amino acid sequence that is at least 90% identical to the amino acid sequence of SEQ ID NO: 2. The Cas9 protein has at least one mutation in an amino acid residue selected from 262, 324, 409, 480, 543, 694, of the amino acid sequence provided in SEQ ID NO: 2 or the corresponding residue of an aligned sequence, and at least one mutation in an amino acid residue selected from 1111, 1135, 1218, 1219, 1322, 1335, and 1337, of the amino acid sequence provided in SEQ ID NO: 2 or the corresponding residue of an aligned sequence. The amino acid sequence of the recombinant Cas9 protein is not identical to the amino acid sequence of a naturally occurring Cas9 protein.

### CROSS REFERENCE TO RELATED APPLICATIONS

This application claims the benefit under 35 USC §119(e) of the priority of U.S. Patent Application No. 62/964,483, filed Jan. 22, 2020. This application is hereby incorporated by reference in its entirety.

### STATEMENT OF GOVERNMENT SUPPORT

This invention was made with government support under HG010099 awarded by the National Institutes of Health and D18AP00053 awarded by DARPA. The government has certain rights in the invention.

### BACKGROUND

Type II CRISPR-Cas9 enzymes are RNA-programmable endonucleases that have been used in diverse DNA-targeting applications, including gene knock-out and knock-in, mutagenesis, gene activation and inhibition, base editing, and CpG methylation (Jinek et al., 2012). Cas9 enzymes, including the most commonly used S. pyogenes Cas9 (Cas9), recognize target DNA sequences that are complementary to their guide RNA spacer and that contain a protospacer adjacent motif (PAM). Although mismatches between the target DNA and a portion of the guide RNA can be tolerated, the presence of the PAM is a strict requirement, which imposes a limit on the number of targetable genomic loci (Hsu et al., 2013). While the availability of PAM sites (such as NGG for Cas9) is typically not a problem for CRISPR-mediated gene knockout because nearly all protein-coding exons can be targeted (Meier et al., 2017), the optimal targeting space for transcriptional modulation (inhibition or activation) is usually smaller, between 50 and 100 nucleotides (Sanson et al., 2018). Other common genome editing tasks, such as homology-directed repair or base editing, require an even narrower window for Cas9 positioning, with the desired target site placed at a precise position from the PAM sequence (e.g. 10-20 nucleotides for homology-directed repair or 13-17 nucleotides for base editing) (Findlay et al., 2014; Komor et al., 2016).

To address this problem, several Cas9 orthologs and other CRISPR nucleases from different bacterial species have been characterized, such as S. aureus Cas9 and Cas12a/Cpf1 (Kim et al., 2016; Ran et al., 2015). However, none of them have a simpler PAM requirement than Cas9. Initial attempts at developing more PAM-flexible Cas9 variants through structure-based design or directed evolution yielded enzymes recognizing slightly altered PAMs but still requiring a three-nucleotide motif (Kleinstiver et al., 2015). Recently, two Cas9 variants capable of recognizing an NG PAM were generated, one through phage-assisted continuous evolution (xCas9), and the other through structure-guided design (Cas9-NG) (Hu et al., 2018; Nishimasu et al., 2018). These Cas9 variants were characterized primarily in terms of their nuclease activity at several endogenous genomic loci, and their relative performance at NG sites was highly variable. One of these PAM-flexible variants, xCas9, led to superior CRISPR activation (CRISPRa) when fused to VP64-p65-Rta (VPR) over wild-type dCas9-VPR, with higher transcriptional activation for all sgRNAs tested. This is presumably due to the directed evolution selection pressure—transcriptional activation and not nuclease activity—used to derive xCas9.

What is needed are Cas9 variants with altered PAM specificity.

### SUMMARY OF THE INVENTION

Provided herein in a first aspect, is a recombinant or engineered Cas9 protein. The Cas9 protein has an amino acid sequence that is at least 90% identical to the amino acid sequence of SEQ ID NO: 2. The Cas9 protein has at least one mutation in an amino acid residue selected from 262, 324, 409, 480, 543, 694, of the amino acid sequence provided in SEQ ID NO: 2 or the corresponding residue of an aligned sequence, and at least one mutation in an amino acid residue selected from 1111, 1135, 1218, 1219, 1322, 1335, and 1337, of the amino acid sequence provided in SEQ ID NO: 2 or the corresponding residue of an aligned sequence. The amino acid sequence of the recombinant Cas9 protein is not identical to the amino acid sequence of a naturally occurring Cas9 protein.

In one embodiment, the mutations are selected from X262T, X324L, X4091, X480K, X543D, X694I, X1111R, X1135V, X1218R, X1219F, X1219V, X1322R, X1335V, and X1337R of the amino acid sequence provided in SEQ ID NO: 2 or the corresponding residue of an aligned sequence, wherein X represents any amino acid. In another embodiment, the mutations are selected from A262T, R324L, S409I, E480K, E543D, M694I, L1111R, D1135V, G1218R, E1219F, E1219V, A1322R, R1335V, and T1337R of the amino acid sequence provided in SEQ ID NO: 2. In one embodiment, the Cas9 protein has the sequence of SEQ ID NO: 1.

In another aspect, a fusion protein is provided. The fusion protein includes a recombinant Cas9 protein as described herein, fused to a heterologous functional domain, with an optional intervening linker, wherein the linker does not interfere with activity of the fusion protein.

In another aspect, a nucleic acid encoding a Cas9 protein or fusion protein as described herein, is provided. In yet another aspect, a vector comprising a nucleic acid encoding a Cas9 protein or fusion protein as described herein, is provided. In another aspect, a host cell comprising nucleic acid encoding a Cas9 protein or fusion protein as described herein, is provided.

In another aspect, a method of altering the genome of a cell is provided. The method includes expressing in the cell, or contacting the cell with, the recombinant Cas9 protein or fusion protein of as described herein, and a guide RNA having a region complementary to a selected portion of the genome of the cell.

In yet another aspect, a method of evaluating a CRISPR-Cas system is provided. The method includes a) obtaining a sgRNA library comprising multiple sgRNA sequences which target sites in the genome, b) cloning said library into a lentiviral plasmid comprising a nucleic acid sequence encoding a Cas protein and, optionally, a barcode, c) producing lentivirus containing said plasmid, d) transducing mammalian cells with said lentivirus, e) culturing said cells for a sufficient time period, and f) evaluating said cells for CRISPR activity.

Other aspects and advantages of the invention will be readily apparent from the following detailed description of the invention.

### BRIEF DESCRIPTION OF THE DRAWINGS

FIGS. 1A-1E show the results of a high-throughput, pooled competition assay for PAM-flexible Cas9 variants. 1A) Gene-specific sgRNA libraries were cloned into lentiviral plasmids containing barcoded Cas9 effectors. After library transduction, K562 cells were sorted by target gene expression level into high- and low-expressing bins and the relative frequency of sgRNA-Cas9 barcode pairs in both bins was compared. 1B) Fold-change of sgRNA representation in cell populations expressing a high level of the target gene compared to low-expressing cells, combined for all three genes tested. Only sgRNAs targeting CDS exons are shown. 1C) Fold-change of sgRNA representation grouped by two- and three-nucleotide PAMs. Statistical significance was determined by comparing fold-change of sgRNAs associated with a particular PAM to a respective non-targeting control using two-tailed Student's t-test with Bonferroni correction for multiple hypothesis testing. Error bars indicate standard error of the mean. Only statistically significant PAM/Cas9 combinations are shown in color. 1D) CD46′ gate, indicated with a dashed line, was set based on K562 autofluorescence. Numbers displayed next to histograms indicate the percentage of cells in CD46^{neg} gate. LV—lentivirus. 1E) Quantification of CD46 knock-out in K562 cell line by lentiviral transduction of Cas9 nucleases and sgRNAs associated with NGG or NGH PAMs. Error bars indicate standard error of the mean.

FIGS. 2A-2E show characterization of indel mutations produced by active Cas9 variants. 2A) Gating strategy for enumeration of K562 cells expressing the wild-type level of CD46 protein. No LV—no lentivirus. 2B) Correlation between the frequency of alleles containing indels and the frequency of cells expressing the wild-type levels of CD46 protein. Dashed lines indicate 95% confidence intervals around the linear regression curve. NGG and NGH sgRNAs are included. r² is the Pearson coefficient of determination. 2C) Relative frequency of deletions and insertions among edited alleles. Each line represents one sgRNA. Only NGG sgRNAs with >5% edited alleles are included. 2 D) Mean deletion and insertion sizes per Cas9 variant. Each data point represents the mean indel size for one sgRNA. Error bars indicate SEM. Only NGG sgRNAs with >5% edited reads are included. 2E) Indel sizes (among edited reads) for each Cas9 variant.

FIGS. 3A-3D show CRISPRi and CRISPRa transcriptional modulation using PAM-flexible Cas9 variants. 3A) Fold-change of sgRNAs targeting the 3 kb region surrounding the primary TSS of CD45 gene. Only sgRNAs associated with an NGG PAM are displayed here (n=123 sgRNAs). The regions with the strongest NGG sgRNA activity (indicated with dashed lines) were used to select sgRNAs (all PAMs) for subsequent analyses. CD45 transcript isoforms (PTPRC-204, PTPRC-215, PRPRC-201, PTPRC-209, PTPRC-206, PTPRC-207) are shown in grey. 3B) Fold-change of sgRNA representation grouped by two- and three-nucleotide PAM categories. Statistical significance was determined by comparing fold-change of sgRNAs associated with a particular PAM to a respective non-targeting control using two-sided Student's t-test with Bonferroni correction. Error bars indicate standard error of the mean. Only statistically significant PAM/Cas9 combinations are shown in color. CRISPRi: n=2,165 sgRNAs; CRISPRa: n=1,980 sgRNAs. 3C) CD45 expression following CRISPR activation in the CD45′ human A375 cell line. Only sgRNAs resulting in >1% CD45^{pos} cells with at least one Cas9 variant are displayed (see FIG. 23 for data from all sgRNAs tested). Mean and individual values from three independent experiments are shown. For NAG and NAA PAMs, only one out of three sgRNAs tested resulted in >1% CD45^{pos} cells. 3D) Comparison of wild-type Cas9 and two PAM-flexible Cas9 variants across all three modalities tested in high-throughput screens. Fold-enrichment was calculated based on the sgRNA frequency in the top bin over bottom bin; fold-depletion was calculated based on the sgRNA frequency in the bottom bin over top bin. Only non-significant comparisons (ns, p>0.05) are indicated; all other differences (between enzymes, within modalities) are significant.

FIGS. 4A-4C show that Cas9-NG mutations partially rescue xCas9 nuclease activity and result in an improved PAM-flexible Cas9 enzyme for transcriptional activation. 4A) Crystal structures of Cas9 mutants. xCas9 mutations are shown on a wild-type Cas9 structure (PDB ID: 4un3, Anders et al., 2014). xCas9-NG mutations are displayed on a Cas9-NG structure (PDB ID: 6ai6, Nishimasu et al., 2018). The sgRNA is shown in black and the target DNA is shown in blue. 4B) Knockout and 4C) activation activity for individual sgRNAs with target sites with the indicated PAMs. Each xCas9 or xCas9-NG experiment was normalized to Cas9-NG on a per-sgRNA basis. Only sgRNAs resulting in >1% knock-out or activation are shown. Non-normalized data for xCas9 and Cas9-NG were displayed in FIGS. 1E and 2C and are included here for comparison with xCas9-NG. ns, p>0.05; *p<0.05, **p<0.01, ****p<0.0001.

FIG. 5 shows that most active sgRNAs have a higher Root-Doench score than least active ones—but only for wild-type Cas9 and target sites with NGG PAMs. The Root-Doench score of the 50% sgRNAs with the highest depletion score in the CRISPR nuclease screen for each Cas9 variant was compared to the 50% sgRNAs with the lowest depletion score. Median and fold difference between median Root-Doench score of the best performing and worst performing sgRNAs are shown. Student's t-test, ns p>0.05, **p<0.01.

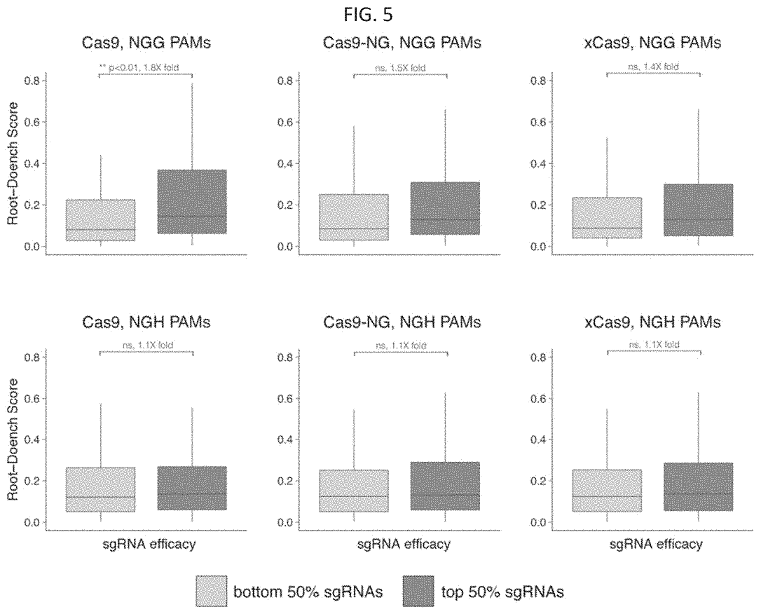

FIG. 6A-6B show the dynamic range of CD45, CD46 and CD55 expression. 6A) K562 cell line was transduced with pooled sgRNA libraries targeting CD45, CD46 and CD55, together with Cas9 effectors (CRISPRi: KRAB-dCas9, KRAB-dCas9-NG, KRAB-dxCas9; CRISPRn: Cas9, Cas9-NG, xCas9). Median fluorescence intensity (MFI) of the cells with the lowest 10% expression level of the target protein was quantified by flow cytometry and compared to the MFI of the corresponding wild-type (WT) population. Standard error of the mean and individual values, corresponding to each Cas9 effector transduced separately, are shown. 6B) K562 cell line was transduced with pooled sgRNA libraries targeting CD45, CD46 and CD55, together with CRISPRa Cas9 effectors: dCas9-VPR, dCas9-NG-VPR, dxCas9-VPR. MFI of the cells with the highest 10% expression level of the target protein was divided by the MFI of the 10% lowest expression level, and compared to the MFI ratio of the wild-type population. Standard error of the mean and individual values, corresponding to each Cas9 effector transduced separately, are shown.

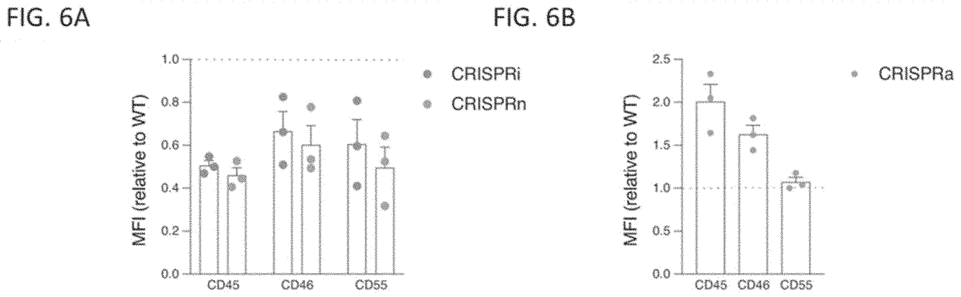

FIG. 7 shows the dynamic range of CD45, CD46 and CD55 expression. FIG. 7 shows representative histograms of target protein expression levels used for arrayed validation. For CRISPRn and CRISPRi, CD45+ CD46+ K562 cell line is shown; for CRISPRa, CD45neg A375 cell line is shown.

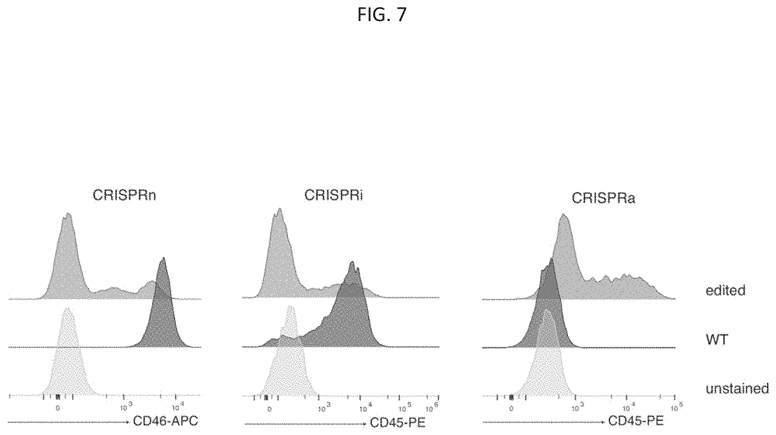

FIG. 8 shows quantification of the expression levels of Cas9 variants. Cas9 expression levels were quantified by western blot using an antibody against the 2A peptide found at the C-terminus of all variants. The expression of Cas9 variants was normalized by the expression level of GAPDH in the same sample. For all three types of Cas9 effectors (CRISPRi, CRISPRa and CRISPRn) the expression levels were normalized to the relative expression level of wild-type Cas9, which was set to 1.

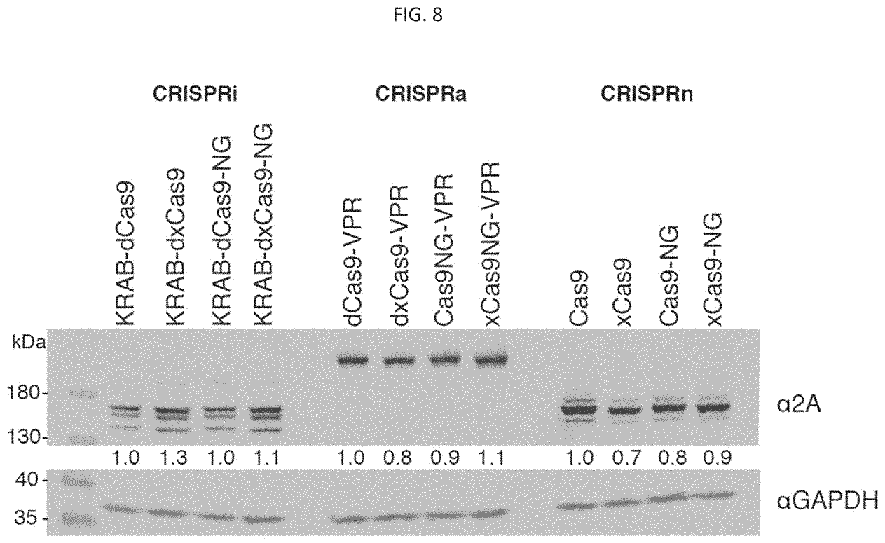

FIG. 9A-9B show the relative abundance of barcodes corresponding to each Cas9 effector in mixed population pre-sort. 9A) Relative frequency of each Cas9 variant in each of the nine cell pools used for screening. Individual values and standard error of the mean are shown. 9B) Read count distributions for every sgRNA with each Cas9 effector shown separately for each screen pool.

FIG. 10 shows a time course of CD46 knock-out by Cas9 variants. CD46+A375 cell line was transduced with lentivirus encoding each of three Cas9 variants and sgRNAs targeting CD46 coding sequences with the indicated PAMs. Following selection, CD46 negative cells were quantified based on the gate set on the unstained population. Standard error of the mean (n=3) is show.

FIG. 11 shows comparison of the delivery methods on perturbation efficiency. Plasmid's encoding KRAB-dCas9 effectors and sgRNAs targeting CD45 TSS were used to produce lentivirus and subsequently transduce K562 cell line. Following selection, CD45 knock-down was quantified in the transduced cells. Plasmids encoding dCas9-VPR effectors and sgRNAs targeting CD45 TSS were directly transfected into A375 cells. Following selection, CD45 over-expression was quantified in the transduced cells. Triplicate values of 2 sgRNAs (NGG) or 4 sgRNAs (NGH) and standard error of the mean are shown. Data displayed here were already shown in FIG. 3C and FIG. 15, and are presented here for comparison purposes. sgRNA sequences are shown in FIG. 17. Paired t-test: ns, p>0.05, **p<0.01, ****p<0.0001.

FIG. 12 shows Cas9-NG outperforms xCas9 at NGH PAM sites across different cell lines. Frequency of cells with modulated expression of target proteins was calculated based on gates set on the negative or positive populations, respectively. For K562 and A375, CRISPR nuclease activity on CD46 gene is shown. For HEK293FT, CRISPR activation of CD45/PTPRC gene is shown. Individual values and standard error of the mean are displayed. sgRNA sequences are listed in FIG. 17. Student's t-test, ns p>0.05, *p<0.05, ***p<0.001.

FIG. 13 shows a comparison of CD45 transcriptional activation efficiency between existing PAM-flexible Cas9 variants and xCas9-NG in HEK-293T cell line. Activation efficiency for xCas9 and xCas9-NG was normalized to Cas9-NG on a per-sgRNA basis. Individual values for each sgRNA are shown with a solid line indicated the mean over sgRNAs with the same PAM. Student's t-test results are shown between xCas9 and Cas9-NG (**p<0.01), xCas9-NG and Cas9-NG (*p<0.05) and xCas9-NG and xCas9 (***p<0.001).

FIG. 14A and 14B show CD45 expression following CRISPR inhibition in CD45⁺ cell line K562. 14A) Reduction of CD45 expression following transduction with Cas9 effectors and sgRNAs targeting the transcription start site of CD45/PTPRC gene (sgRNA sequences are shown in Table S2). For each Cas9 variant and sgRNA, the median fluorescence intensity (MFI) of CD45 staining was normalized by dividing the MFI of corresponding non-targeting sgRNAs by targeting sgRNAs. Individual values and standard error of the mean (3 replicates per sgRNA) are shown. 14B) Knock-down activity for individual sgRNAs with target sites with the indicated PAMs. For each sgRNA, mean of three replicates is shown. Error bars indicate standard error of the mean. Two-sided t-test: ns, p>0.05, **p<0.01, ***p<0.001, ****p<0.0001.

FIG. 15A-15D shows targetable sites with NG and NG+NAD PAMs in all protein-coding genes in the human genome. Targetable sites when using additional PAMs (beyond NGG) in 15A) coding exons of 18,544 human protein-coding genes, 15B) near transcription start sites in optimal CRISPRi regions, 15C) near transcription start sites in optimal CRISPRa regions, and 15D) 202,000 DNAse I hypersensitivity (HS) sites in K562 cell line. For example, in CDS exons, there is a 3.7× mean increase in target sites with NG PAMs and a 6.6× mean increase with NAD and NG PAMs. All increases are given as fold-change over targeting NGG PAM sites alone.

FIG. 16 shows the composition of the CRISPR libraries for each screen.

FIG. 17 shows the sgRNA sequences used.

FIG. 18 shows PCR primer sequences. Stagger (between 1 and 9 nucleotides) is shown in brackets; examples of 8 nucleotide i7 and i5 barcodes for sample multiplexing are shown in bold. A full set of PCR2 primers (with multiple barcodes and stagger) is available for download here (readout primers v. July 2014): http://sanjanalab.org/lib.html

FIG. 19A-19D shows lentiviral vector design and screen gating strategy. 19A) Lentiviral expression vector for Cas9 and sgRNA expression, based on the lentiCRISPRv2 system (Sanjana et al., 2014). sgRNA scaffold was modified for improved activity (Chen et al, 2013)²⁹. A six nucleotide barcode was inserted between the sgRNA scaffold and the EFS promoter to enable identification of Cas9 variant and effector. For transcriptional repression, KRAB domain was fused to catalytically inactive Cas9 (dCas9, D10A, H840A) on N-terminus; for transcriptional activation, VP64-p65-Rta (VPR) complex was fused to catalytically inactive Cas9 (dCas9, D10A, H840A) on C-terminus. In all cases Cas9 sequences were codon optimized for human expression and contained a C-terminal nuclear localization signal (NLS). Long terminal repeat (LTR), psi packaging signal (psi+), rev response element (RRE), central polypurine tract (cPPT), elongation factor-1a short promoter (EFS), 2A self-cleaving peptide (P2A), puromycin selection marker (puro), posttranscriptional regulatory element (WPRE). 19B) Gating strategy for flow cytometry analyses and sorting. The cells were separated from debris based on forward (FSC) and side scatter (SSC) properties, followed by doublet and dead cell exclusion. 19C) Surface expression of CD45, CD46 and CD55 in K562 cell line. 19D) Sorting gates for CRISPR nuclease, repression and activation pools in K562 cells. Representative histograms for one of the genes (CD55) are shown.

FIGS. 20A-20B show sgRNA count correlations between Cas9 enzymes within the same pool before sorting. 20A) Representative scatterplots for nuclease, repressor and activator pools. r indicates Pearson correlation coefficient. 20B) Pearson correlation coefficient of normalized read counts for each Cas9 combination within the same pool (modality+gene).

FIGS. 21A-21B show nuclease CRISPR competition screen for CD45, CD46, and CD55. 21A) Fold-change of CDS-targeting sgRNAs for each Cas9 variant in cell populations expressing high levels of target gene (CD45: n=1,027 sgRNAs; CD46: n=1,025 sgRNAs; CD55: n=1,122 sgRNAs) over low-expressing cells is shown. 21B) Fold-change of sgRNAs targeting between 1.5 kb and 0.5 kb upstream of the TSS in cell populations expressing high level of target gene (CD45: n=655 sgRNAs; CD46: n=725 sgRNAs; CD55: n=781 sgRNAs) over low-expressing cells is shown. Median of non-targeting (NT) sgRNAs was used to normalize fold-change for each enzyme.

FIGS. 22A-22D shows: High-throughput CRISPR transcriptional modulation screen at CD46 and CD55. Fold-change of sgRNAs targeting the 3 kb region surrounding the primary TSS of CD46 (FIG. 22A and FIG. 22B) and CD55 (FIG. 22C and FIG. 22D) genes. Only sgRNAs associated with the NGG PAM are displayed (CD46: n=393 sgRNAs; CD55: n=400 sgRNAs). The regions with strongest NGG sgRNA activity (indicated with dashed lines) were used to select sgRNAs for subsequent analyses. Collapsed gene models of CD46 and CD55 isoforms (Ensembl CD46-201, CD46-206; CD55-204, CD55-203, CD55-206, CD55-201, CD55-213, CD55-202) are shown in grey.

FIGS. 23A-23B show transcriptional modulation CRISPR competition screen results for sgRNAs targeting the optimal region surrounding the TSS. Fold-change of sgRNAs in sorted cell populations expressing high level of target gene over low-expressing cells is shown (FIG. 23A—CRISPR inhibition, FIG. 23B—CRISPR activation). The median of the non-targeting sgRNAs was used to normalize fold-change for each Cas9 variant. Only sgRNAs targeting the optimal region surrounding the gene TSS are included in the analysis (CRISPR inhibition, CD45: n=624 sgRNAs; CD46: n=577 sgRNAs; CD55: n=946 sgRNAs; CRISPR activation, CD45: n=670 sgRNAs; CD46: n=506 sgRNAs; CD55: n=804 sgRNAs).

FIGS. 24A-24D shows transcriptional modulation CRISPR competition screen results for CDS targeting sgRNAs. Fold-change of sgRNAs in cell populations expressing high level of target gene over low-expressing cells is shown. Median of non-targeting (NT) sgRNAs was used to normalize fold-change for each enzyme. Only sgRNAs targeting CDS exons are included in the analysis. FIG. 24A) CRISPR inhibition screen and FIG. 24B) CRISPR activation screen—all three genes together. FIG. 24C) CRISPR inhibition screen and FIG. 24D) CRISPR activation screen split by gene: CD45: n=984 sgRNAs; CD46: n=949 sgRNAs; CD55: n=924 sgRNAs

FIG. 25A-25B shows CD45 expression following CRISPR activation in CD45^{neg} A375 cell line. 25A) Representative histograms of CD45 staining for each PAM category. Dashed line indicates CD45^{pos} gate; numbers on histograms correspond to the percentage of cells in CD45^{pos} gate. 25B) CD45 positive cells after CRISPRa transduction. Mean and individual values from three independent experiments are shown (n=23 sgRNAs, each tested with all three Cas9 variants).

FIG. 26A-26B shows CD45 expression following CRISPR activation in CD45^{neg} HEK-293FT cell line. 26A) Representative histograms of CD45 staining for each PAM category. Dashed line indicates CD45^{pos} gate; numbers on histograms correspond to the percentage of cells in CD45^{pos} gate. 26B) CD45 positive cells after CRISPRa transduction. Two (NGG) or three (all other PAM categories) sgRNAs per PAM were tested in a single experiment. n=23 sgRNAs, each tested with all three Cas9 variants.

### DETAILED DESCRIPTION OF THE INVENTION

A key limitation of the commonly-used CRISPR enzyme S. pyogenes Cas9 is the strict requirement of an NGG protospacer-adjacent motif (PAM) at the target site, which reduces the number of accessible genomic loci. This constraint can be limiting for genome editing applications that require precise Cas9 positioning. Recently, two Cas9 variants with a relaxed PAM requirement (NG) have been developed (xCas9 and Cas9-6NG) but their activity has been measured at only a small number of endogenous sites. See, US Patent Publication No. 2019/0106687 and US Patent Publication No. 2019/0225955, both of which are incorporated herein by reference.

Given the utility of PAM-flexible Cas9 enzymes for precise genome engineering, we designed an unbiased, massively-parallel competition assay to compare Cas9 enzyme variants at thousands of target sites in the human genome. We benchmarked both PAM-flexible enzymes head-to-head with Cas9 for nuclease-driven loss-of-function, gene activation and gene repression. Across all 3 modalities, we found that PAM flexibility comes at the cost of markedly lower activity. Wild-type Cas9 outperformed both PAM-flexible variants at NGG sites for every modality tested. At NGH PAMs (H=A, C or T), we found that Cas9-NG is universally better than xCas9 and that xCas9 is often indistinguishable from the wild-type enzyme.

We were able to partially rescue xCas9 nuclease activity by adding Cas9-NG mutations to create a new Cas9 variant, xCas9-NG. For gene activation, we found that xCas9-NG outperforms both xCas9 and Cas9-NG at both NGG and NGH PAMs. We expect that this novel PAM-flexible Cas9 will be useful for a multitude of genome-engineering applications where precise Cas9 positioning is required.

Recombinant Cas9 Variants with Altered PAM Specificities

Provided herein are recombinant or engineered Cas9 variants. The Cas9 variants described herein greatly increase the range of target sites accessible by wild-type Cas9. The Streptococcus pyogenes (sp) Cas9 wild type sequence is as follows, and is set forth in SEQ ID NO: 2 (Uniprot Q99ZW2-1):

| | |
|---|---|
| MDKKYSIGLD IGTNSVGWAV ITDEYKVPSK KFKVLGNTDR | 60 |
| HSIKKNLIGA LLFDSGETAE | |
| ATRLKRTARR RYTRRKNRIC YLQEIFSNEM AKVDDSFFHR | 120 |
| LEESFLVEED KKHERHPIFG | |
| NIVDEVAYHE KYPTIYHLRK KLVDSTDKAD LRLIYLALAH | 180 |
| MIKFRGHFLI EGDLNPDNSD | |
| VDKLFIQLVQ TYNQLFEENP INASGVDAKA ILSARLSKSR | 240 |
| RLENLIAQLP GEKKNGLFGN | |
| LIALSLGLTP NFKSNFDLAE DAKLQLSKDT YDDDLDNLLA | 300 |
| QIGDQYADLF LAAKNLSDAI | |
| LLSDILRVNT EITKAPLSAS MIKRYDEHHQ DLTLLKALVR | 360 |
| QQLPEKYKEI FFDQSKNGYA | |
| GYIDGGASQE EFYKFIKPIL EKMDGTEELL VKLNREDLLR | 420 |
| KORTFDNGSI PHQIHLGELH | |
| AILRRQEDFY PFLKDNREKI EKILTFRIPY YVGPLARGNS | 480 |
| RFAWMTRKSE ETITPWNFEE | |
| VVDKGASAQS FIERMTNFDK NLPNEKVLPK HSLLYEYFTV | 540 |
| YNELTKVKYV TEGMRKPAFL | |
| SGEQKKAIVD LLFKTNRKVT VKQLKEDYFK KIECFDSVEI | 600 |
| SGVEDRFNAS LGTYHDLLKI | |
| IKDKDFLDNE ENEDILEDIV LTLTLFEDRE MIEERLKTYA | 660 |
| HLFDDKVMKQ LKRRRYTGWG | |
| RLSRKLINGI RDKQSGKTIL DFLKSDGFAN RNFMQLIHDD | 720 |
| SLTFKEDIQK AQVSGQGDSL | |
| HEHIANLAGS PAIKKGILQT VKVVDELVKV MGRHKPENIV | 780 |
| IEMARENQTT QKGQKNSRER | |
| MKRIEEGIKE LGSQILKEHP VENTQLQNEK LYLYYLQNGR | 840 |
| DMYVDQELDI NRLSDYDVDH | |
| IVPQSFLKDD SIDNKVLTRS DKNRGKSDNV PSEEVVKKMK | 900 |
| NYWRQLLNAK LITQRKFDNL | |
| TKAERGGLSE LDKAGFIKRQ LVETRQITKH VAQILDSRMN | 960 |
| TKYDENDKLI REVKVITLKS | |
| KLVSDFRKDF QFYKVREINN YHHAHDAYLN AVVGTALIKK | 1020 |
| YPKLESEFVY GDYKVYDVRK | |
| MIAKSEQEIG KATAKYFFYS NIMNFFKTEI TLANGEIRKR | 1080 |
| PLIETNGETG EIVWDKGRDF | |
| ATVRKVLSMP QVNIVKKTEV QTGGFSKESI LPKRNSDKLI | 1140 |
| ARKKDWDPKK YGGFDSPTVA | |
| YSVLVVAKVE KGKSKKLKSV KELLGITIME RSSFEKNPID | 1200 |
| FLEAKGYKEV KKDLIIKLPK | |
| YSLFELENGR KRMLASAGEL QKGNELALPS KYVNFLYLAS | 1260 |
| HYEKLKGSPE DNEQKQLFVE | |
| QHKHYLDEII EQISEFSKRV ILADANLDKV LSAYNKHRDK | 1320 |
| PIREQAENII HLFTLTNLGA | |
| PAAFKYFDTT IDRKRYTSTK EVLDATLIHQ SITGLYETRI | 1368 |
| DLSQLGGD | |

In one aspect, the recombinant Cas9 protein (also called Cas9 variant) has a variation in at least one amino acid selected from residues 262, 324, 409, 480, 543, 694, of the amino acid sequence provided in SEQ ID NO: 2 or the corresponding residue of an aligned sequence, and at least one mutation in an amino acid residue selected from 1111, 1135, 1218, 1219, 1322, 1335, and 1337 of the amino acid sequence provided in SEQ ID NO: 2 or the corresponding residue of an aligned sequence. The recombinant or engineered Cas9 protein is not identical to the amino acid sequence of a naturally occurring Cas9 protein.

In some embodiments, the Cas9 variant is at least 80%, e.g., at least 85%, 90%, 95%, 96%, 97%, 98% or 99% identical to the amino acid sequence of SEQ ID NO: 2, e.g., has differences at up to 1%, 2,%, 3%, 4%, 5%, 10%, 15%, or 20% of the residues of SEQ ID NO: 2, replaced, e.g., with conservative mutations. In one embodiment, the Cas9 protein is at least 95% identical to SEQ ID NO: 2. In preferred embodiments, the variant retains desired activity of the parent, e.g., the nuclease activity (except where the parent is a nickase or a dead Cas9), and/or the ability to interact with a guide RNA and target DNA), although not necessarily at the same level.

To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The residues at corresponding amino acid positions are then compared. When a position in the first sequence is occupied by the same residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned using the BLAST algorithm and the default parameters.

In some embodiments, the SpCas9 variants include mutations at one, two, three, four, five, or all six of the following positions: 262, 324, 409, 480, 543, and 694 of the amino acid sequence provided in SEQ ID NO: 2 or the corresponding residue of an aligned sequence. In some embodiments, the SpCas9 variants include mutations at one, two, three, four, five, six, or all seven of the following positions: 1111, 1135, 1218, 1219, 1322, 1335, and 1337 of the amino acid sequence provided in SEQ ID NO: 2 or the corresponding residue of an aligned sequence. In some embodiments, the SpCas9 variants include mutations at two, three, four, five, six, seven, eight, nine, ten, eleven, twelve, or all thirteen of the following positions: 262, 324, 409, 480, 543, 694, 1111, 1135, 1218, 1219, 1322, 1335, and 1337 of the amino acid sequence provided in SEQ ID NO: 2 or the corresponding residue of an aligned sequence.

In some embodiments, the mutations are selected from X262T, X324L, X4091, X480K, X543D, X694I, X1111R, X1135V, X1218R, X1219F, X1219V, X1322R, X1335V, and X1337R of the amino acid sequence provided in SEQ ID NO: 2 or the corresponding residue of an aligned sequence, wherein X represents any amino acid. However, substitutions for the mutated residue may be selected, especially amongst conservative residues. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. For example X262T or X262F.

In another embodiment, the mutations are selected from A262T, R324L, S409I, E480K, E543D, M694I, L1111R, D1135V, G1218R, E1219V, A1322R, R1335V, and T1337R of the amino acid sequence provided in SEQ ID NO: 2 or the corresponding residue of an aligned sequence. In another embodiment, the mutations are selected from A262T, R324L, S409I, E480K, E543D, M694I, L1111R, D1135V, G1218R, E1219F, A1322R, R1335V, and T1337R of the amino acid sequence provided in SEQ ID NO: 2 or the corresponding residue of an aligned sequence. In one embodiment, the Cas9 protein has the sequence of SEQ ID NO: 1, sometimes referred to herein as xCas9-NG. In another embodiment, the Cas9 protein has the sequence of SEQ ID NO: 3.

xCas9-NG
SEQ ID NO: 1
MDKKYSIGLD IGTNSVGWAV ITDEYKVPSK KFKVLGNTDR
HSIKKNLIGA LLFDSGETAEATRLKRTARR RYTRRKNRIC
YLQEIFSNEM AKVDDSFFHR LEESFLVEED
KKHERHPIFGNIVDEVAYHE KYPTIYHLRK KLVDSTDKAD
LRLIYLALAH MIKFRGHFLI EGDLNPDNSDVDKLFIQLVQ
TYNQLFEENP INASGVDAKA ILSARLSKSR RLENLIAQLP
GEKKNGLFGNLIALSLGLTP NFKSNFDLAE DTKLQLSKDT
YDDDLDNLLA QIGDQYADLF LAAKNLSDAILLSDILRVNT
EITKAPLSAS MIKLYDEHHQ DLTLLKALVR QQLPEKYKEI
FFDQSKNGYAGYIDGGASQE EFYKFIKPIL EKMDGTEELL
VKLNREDLLR KQRTFDNGII PHQIHLGELHAILRRQEDFY
PFLKDNREKI EKILTFRIPY YVGPLARGNS RFAWMTRKSE
ETITPWNFEKVVDKGASAQS FIERMTNFDK NLPNEKVLPK
HSLLYEYFTV YNELTKVKYV TEGMRKPAFLSGDQKKAIVD
LLFKTNRKVT VKQLKEDYFK KIECFDSVEI SGVEDRFNAS
LGTYHDLLKIIKDKDFLDNE ENEDILEDIV LTLTLFEDRE
MIEERLKTYA HLFDDKVMKQ LKRRRYTGWGRLSRKLINGI
RDKQSGKTIL DFLKSDGFAN RNFIQLIHDD SLTFKEDIQK
AQVSGQGDSLHEHIANLAGS PAIKKGILQT VKVVDELVKV
MGRHKPENIV IEMARENQTT QKGQKNSRERMKRIEEGIKE
LGSQILKEHP VENTQLQNEK LYLYYLQNGR DMYVDQELDI
NRLSDYDVDHIVPQSFLKDD SIDNKVLTRS DKNRGKSDNV
PSEEVVKKMK NYWRQLLNAK LITQRKFDNLTKAERGGLSE
LDKAGFIKRQ LVETRQITKH VAQILDSRMN TKYDENDKLI
REVKVITLKS KLVSDFRKDF QFYKVREINN YHHAHDAYLN
AVVGTALIKK YPKLESEFVY GDYKVYDVRK MIAKSEQEIG
KATAKYFFYS NIMNFFKTEI TLANGEIRKR PLIETNGETG
EIVWDKGRDF ATVRKVLSMP QVNIVKKTEV QTGGFSKESI
RPKRNSDKLI ARKKDWDPKK YGGFVSPTVA YSVLVVAKVE
KGKSKKLKSV KELLGITIME RSSFEKNPID FLEAKGYKEV
KKDLIIKLPK YSLFELENGR KRMLASARFL QKGNELALPS
KYVNFLYLAS HYEKLKGSPE DNEQKQLFVE QHKHYLDEII
EQISEFSKRV ILADANLDKV LSAYNKHRDK PIREQAENII
HLFTLTNLGA

The Cas9 proteins exemplified herein are derived from S. pyogenes (Sp), which wild type sequence can be found in SEQ ID NO: 2. This wild type sequence is used herein, for simplicity as the base sequence on which the variants are described. However, all of the Cas9 variants described herein can be utilized with other previously described improvements to the Cas9 platform (e.g., truncated sgRNAs (Tsai et al., Nat Biotechnol 33, 187-197 (2015); Fu et al., Nat Biotechnol 32, 279-284 (2014)), nickase mutations (Mali et al., Nat Biotechnol 31, 833-838 (2013); Ran et al., Cell 154, 1380-1389 (2013)), dimeric FokI-dCas9 fusions (Guilinger et al., Nat Biotechnol 32, 577-582 (2014); Tsai et al., Nat Biotechnol 32, 569-576 (2014)); and high-fidelity variants (Kleinstiver et al. Nature 2016). Each of these documents is incorporated herein by reference. That is, in one embodiment, the starting Cas9 is a variant from the wild type Cas9 shown in SEQ ID NO: 2.

In some embodiments, in addition to the mutations described above, the Cas9 variants also include mutations at one of the following amino acid positions, which reduce or destroy the nuclease activity of the Cas9: D10, E762, D839, H983, or D986 and H840 or N863, e.g., D10A/D10N and H840A/H840N/H840Y, to render the nuclease portion of the protein catalytically inactive (also referred to as dead Cas9 or dCas9)(SEQ ID NO:5). Substitutions at these positions could be alanine (as they are in Nishimasu al., Cell 156, 935-949 (2014), which is incorporated by reference herein), or other residues, e.g., glutamine, asparagine, tyrosine, serine, or aspartate, e.g., E762Q, H983N, H983Y, D986N, N863D, N863S, or N863H (see WO 2014/152432). In some embodiments, the variant includes mutations at D10A or H840A (which creates a single-strand nickase). The sequence of Cas9D10A is shown in SEQ ID NO: 4. In one embodiment, the Cas9 variant has at least one mutation in a residue selected from 262, 324, 409, 480, 543, 694, of the amino acid sequence provided in SEQ ID NO: 4, and at least one mutation in an amino acid residue selected from 1111, 1135, 1218, 1219, 1322, 1335, and 1337 of the amino acid sequence provided in SEQ ID NO: 4. In another embodiment, the Cas9 variant has at least one mutation in a residue selected from 262, 324, 409, 480, 543, 694, of the amino acid sequence provided in SEQ ID NO: 4, and at least one mutation in an amino acid residue selected from 1111, 1135, 1218, 1219, 1322, 1335, and 1337 of the amino acid sequence provided in SEQ ID NO: 4.

Also provided herein are isolated nucleic acids encoding the Cas9 variants, vectors comprising the isolated nucleic acids, optionally operably linked to one or more regulatory domains for expressing the variant proteins, and host cells, e.g., mammalian host cells, comprising the nucleic acids, and optionally expressing the variant proteins.

The variants described herein can be used for altering the genome of a cell; the methods generally include expressing the variant proteins in the cells, along with a guide RNA having a region complementary to a selected portion of the genome of the cell. Methods for selectively altering the genome of a cell are known in the art, see, e.g., U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; US20140170753; Makarova et al., “Evolution and classification of the CRISPR-Cas systems” 9(6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20(9) Molecular Therapy 1658-1660 (September 2012); U.S. Appl. No. 61/652,086, filed May 25, 2012; Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

The variant proteins described herein can be used in place of the Cas9 proteins described in the foregoing references with guide RNAs that target sequences that have the following PAM sequences: NGG and NGH, where N is A, G, C, or T, and where H is A, C, or T. As described herein, xCas9-NG has been shown to outperform previously described variants xCas9 and Cas9-NG at both NGG and NGH PAMs. In one embodiment, the PAM has the following sequence: AGG, GGG, CGG, TGG, AGA, GGA, CGA, TGA, AGC, GGC, CGC, TGC, AGT, GGT, CGT, or TGT.

Also provided herein are fusion proteins comprising the Cas9 variants described herein. In one embodiment, the Cas9 protein is fused to a heterologous functional domain, with an optional intervening linker, wherein the linker does not interfere with activity of the fusion protein. The variants described herein can be used in fusion proteins in place of the wild-type Cas9 or other Cas9 mutations (such as the dCas9 or Cas9 nickase described above) as known in the art, e.g., a fusion protein with a heterologous functional domain as described in WO 2014/124284. For example, the N or C terminus of the Cas9 variant can be fused to a heterologous functional domain.

In one embodiment, the heterologous functional domain is a transcriptional activation domain. Transcriptional activation domains include VP16, VP64, rTA, NF-κB p65, or the composite VPR (VP64-p65-rTA). In another embodiment, the functional domain is a transcriptional silencer or transcriptional repression domain. Transcriptional repression domains include Krueppel-associated box (KRAB) domain, ERF repressor domain (ERD), and mSin3A interaction domain (SID), and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998), which is incorporated herein by reference. Transcriptional silencers include Heterochromatin Protein 1 (HP1, also known as swi6), e.g., HP1α or HP1β. Other heterologous functional domains include proteins or peptides that could recruit long non-coding RNAs (lncRNAs) fused to a fixed RNA binding sequence such as those bound by the MS2 coat protein, endoribonuclease Csy4, or the lambda N protein. In another embodiment, the functional domain is an enzyme that that modifies the methylation state of DNA such as a DNA methyltransferase (DNMT) or TET protein. In another embodiment, the functional domain is an enzyme that modifies a histone subunit, such as histone acetyltransferases (HAT), histone deacetylases (HDAC), histone methyltransferases (e.g., for methylation of lysine or arginine residues) and histone demethylases (e.g., for demethylation of lysine or arginine residues).

In some embodiments, the heterologous functional domain is a base editor. Suitable base editors include a deaminase that modifies cytosine DNA bases, e.g., a cytidine deaminase from the apolipoprotein B mRNA-editing enzyme, catalytic polypeptide-like (APOBEC) family of deaminases, including APOBEC1, APOBEC2, APOBEC3A, APOBEC3B, APOBEC3C, APOBEC3D/E, APOBEC3F, APOBEC3G, APOBEC3H, and APOBEC4 (see, e.g., Yang et al., J Genet Genomics. 2017 Sep. 20; 44(9):423-437); activation-induced cytidine deaminase (AID), e.g., activation induced cytidine deaminase (AICDA); cytosine deaminase 1 (CDA1) and CDA2; and cytosine deaminase acting on tRNA (CDAT). Each of these documents is incorporated by reference herein. The following provides exemplary sequences with GenBank Accession Nos.; other sequences can also be used.

hAID/AICDA NM_020661.3 isoform 1 NP 065712.1 variant 1 NM_020661.3 isoform 2 NP 065712.1 variant 2

APOBEC1 NM_001644.4 isoform a NP_001635.2 variant 1 NM_005889.3 isoform b NP_005880.2 variant 3

APOBEC2 NM_006789.3 NP_006780.1

APOBEC3A NM 145699.3 isoform a NP_663745.1 variant 1 NM_001270406.1 isoform b NP_001257335.1 variant 2

APOBEC3B NM_004900.4 isoform a NP_004891.4 variant 1 NM_001270411.1 isoform b NP_001257340.1 variant 2

APOBEC3C NM_014508.2 NP_055323.2

APOBEC3D/E NM_152426.3 NP_689639.2

APOBEC3F NM 145298.5 isoform a NP_660341.2 variant 1 NM_001006666.1 isoform b NP_001006667.1 variant 2

APOBEC3G NM_021822.3 (isoform a) NP_068594.1 (variant 1)

APOBEC3H NM_001166003.2 NP_001159475.2 (variant SV-200)

APOBEC4 NM_203454.2 NP_982279.1 CDA1* NM_127515.4 NP_179547.1

In some embodiments, the heterologous functional domain is a deaminase that modifies adenosine DNA bases, e.g., the deaminase is an adenosine deaminase 1 (ADA1), ADA2; adenosine deaminase acting on RNA 1 (ADAR1), ADAR2, ADAR3 (see, e.g., Savva et al., Genome Biol. 2012 Dec. 28; 13(12):252); adenosine deaminase acting on tRNA 1 (ADAT1), ADAT2, ADAT3 (see Keegan et al., RNA. 2017 September; 23(9):1317-1328 and Schaub and Keller, Biochimie. 2002 August; 84(8):791-803); and naturally occurring or engineered tRNA-specific adenosine deaminase (TadA) (see, e.g., Gaudelli et al., Nature. 2017 Nov. 23; 551(7681):464-471) (NP_417054.2 (Escherichia coli str. K-12 substr. MG1655); See, e.g., Wolf et al., EMBO J. 2002 Jul. 15; 21(14):3841-51). Each of these documents is incorporated by reference herein. The following provides exemplary sequences with GenBank Accession Nos; other sequences can also be used.

ADA (ADA1) NM_000022.3 variant 1 NP_000013.2 isoform 1

ADA2 NM_001282225.1 NP_001269154.1

ADAR NM_001111.4 NP_001102.2

ADAR2 NM_001112.3 variant 1 NP_001103.1 isoform 1 (ADARB1)

ADAR3 NM_018702.3 NP_061172.1 (ADARB2)

ADAT1 NM_012091.4 variant 1 NP_036223.2 isoform 1

ADAT2 NM_182503.2 variant 1 NP_872309.2 isoform 1

ADAT3 NM_138422.3 variant 1 NP_612431.2 isoform 1

In another embodiment, the heterologous functional domain is a prime editor. Prime editors have recently been shown to insert genetic information into a specified DNA site using a catalytically impaired Cas9 endonuclease fused to an engineered reverse transcriptase, programmed with a prime editing guide RNA (pegRNA) that both specifies the target site and encodes the desired edit. Thus, in one embodiment, the Cas9 variant is based on a Cas9 nickase, and is fused to a reverse transcriptase domain. This fusion protein then complexes with the guide RNA (pegRNA) to form the Prime Editing complex. In another embodiment, the heterologous functional domain is a reverse transcriptase. See, Anzalone et al, Search-and-replace genome editing without double-strand breaks or donor DNA, Nature, 576:149-157 (Dec. 5, 2019), which is incorporated herein by reference.

In some embodiments, the heterologous functional domain is an enzyme, domain, or peptide that inhibits or enhances endogenous DNA repair or base excision repair (BER) pathways. Such enzymes, domains, or peptides include thymine DNA glycosylase (TDG; GenBank Acc Nos. NM_003211.4 (nucleic acid) and NP_003202.3 (protein)) or uracil DNA glycosylase (UDG, also known as uracil N-glycosylase, or UNG; GenBank Acc Nos. NM_003362.3 (nucleic acid) and NP_003353.1 (protein)) or uracil DNA glycosylase inhibitor (UGI) that inhibits UNG mediated excision of uracil to initiate BER (see, e.g., Mol et al., Cell 82, 701-708 (1995); Komor et al., Nature. 2016 May 19; 533(7603)); or DNA end-binding proteins such as Gam, which is a protein from the bacteriophage Mu that binds free DNA ends, inhibiting DNA repair enzymes and leading to more precise editing (less unintended base edits). See, e.g., Komor et al., Sci Adv. 2017 Aug. 30; 3(8):eaao4774. See, e.g., Komor et al., Nature. 2016 May 19; 533(7603):420-4; Nishida et al., Science. 2016 Sep. 16; 353(6305). pii: aaf8729; Rees et al., Nat Commun. 2017 Jun. 6; 8:15790; or Kim et al., Nat Biotechnol. 2017 April; 35(4):371-376) as are known in the art can also be used. Each of these documents is incorporated by reference herein. In another embodiment, the heterologous functional domain is a recombinase. In another embodiment, the heterologous functional domain is a nickase.

A number of sequences for domains that catalyze hydroxylation of methylated cytosines in DNA are known. Exemplary proteins include the Ten-Eleven-Translocation (TET)1-3 family, enzymes that converts 5-methylcytosine (5-mC) to 5-hydroxymethylcytosine (5-hmC) in DNA.

Sequences for human TET1-3 are known in the art and are shown below with GenBank Accession Nos:

TET1 NP_085128.2 NM_030625.2

TET2* NP_001120680.1 (var 1) NM_001127208.2 NP_060098.3 (var 2) NM_017628.4

TET3 NP_659430.1 NM_144993.1

*Variant (1) represents the longer transcript and encodes the longer isoform (a). Variant (2) differs in the 5′ UTR and in the 3′ UTR and coding sequence compared to variant 1. The resulting isoform (b) is shorter and has a distinct C-terminus compared to isoform a.

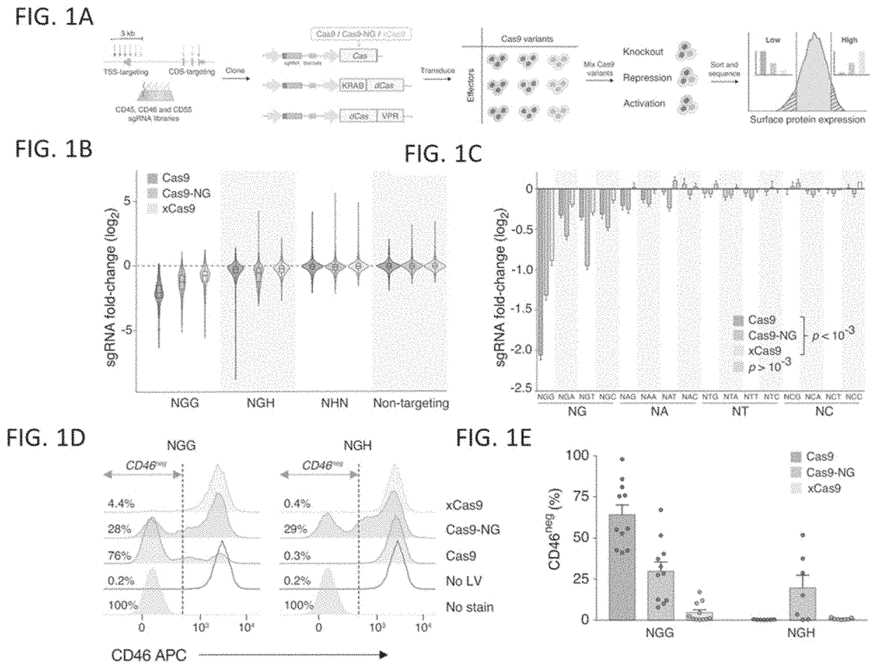

In some embodiments, all or part of the full-length sequence of the catalytic domain can be included, e.g., a catalytic module comprising the cysteine-rich extension and the 2OGFeDO domain encoded by 7 highly conserved exons, e.g., the Tea catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11):1698-710. Epub 2009 Jun. 27, for an alignment illustrating the key catalytic residues in all three Tet proteins, and the supplementary materials thereof for full length sequences (see, e.g., seq 2c); in some embodiments, the sequence includes amino acids 1418-2136 of Tea or the corresponding region in Tet2/3. Other catalytic modules can be from the proteins identified in Iyer et al., 2009.

In some embodiments, the heterologous functional domain is a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the dCas9 gRNA targeting sequences. For example, a dCas9 variant fused to MS2 coat protein, endoribonuclease Csy4, or lambda N can be used to recruit a long non-coding RNA (lncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCas9 variant binding site using the methods and compositions described herein. In some embodiments, the Csy4 is catalytically inactive. In some embodiments, the Cas9 variant, preferably a dCas9 variant, is fused to FokI as described in WO 2014/204578.

In some embodiments, the fusion proteins include a linker between the Cas9 variant and the heterologous functional domain. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:2) or GGGGS (SEQ ID NO:3), e.g., two, three, four, or more repeats of the GGGS (SEQ ID NO:2) or GGGGS (SEQ ID NO:3) unit. Other linker sequences can also be used.

Delivery and Expression Systems

To use the Cas9 variants described herein, it may be desirable to express them from a nucleic acid that encodes them. In another aspect, provided herein is a nucleic acid encoding any of the Cas9 variants or fusion proteins described herein. In one embodiment, the nucleic acid encoding the Cas9 variant is cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the Cas9 variant for production of the Cas9 variant. The nucleic acid encoding the Cas9 variant can also be cloned into an expression vector, for administration to a plant cell, animal cell, preferably a mammalian cell or a human cell, fungal cell, bacterial cell, or protozoan cell.

To obtain expression, a sequence encoding a Cas9 variant is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the Cas9 variant is to be administered in vivo for gene regulation, either a constitutive or an inducible promoter can be used, depending on the particular use of the Cas9 variant. In addition, a preferred promoter for administration of the Cas9 variant can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains an expression cassette that contains all the additional elements required for the expression of the nucleic acid in host cells, either prokaryotic or eukaryotic. A typical expression cassette thus contains a promoter operably linked, e.g., to the nucleic acid sequence encoding the Cas9 variant, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the Cas9 variant, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the Cas9 variants can include RNA Pol III promoters to drive expression of the guide RNAs, e.g., the H1, U6 or 7SK promoters. These human promoters allow for expression of Cas9 variants in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters. The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the Cas9 variant.

Alternatively, the methods can include delivering the Cas9 variant protein and guide RNA together, e.g., as a complex. For example, the Cas9 variant and gRNA can be overexpressed in a host cell and purified, then complexed with the guide RNA (e.g., in a test tube) to form a ribonucleoprotein (RNP), and delivered to cells. In some embodiments, the variant Cas9 can be expressed in and purified from bacteria through the use of bacterial Cas9 expression plasmids. For example, His-tagged variant Cas9 proteins can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you'd get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

### Methods of Evaluating CRISPR-Cas Systems

As the CRISPR technology landscape develops, it is useful to have a means for evaluating various Cas enzyme variants and other variations to the CRISPR machinery. Thus, provided herein is a method of evaluating a CRISPR-Cas system. The method includes obtaining a guide RNA library which includes multiple gRNA sequences which target sites in the genome. The library is cloned into a plasmid comprising a nucleic acid sequence encoding a Cas protein and, optionally, a barcode, and the virus is produced. Host cells, preferably mammalian cells, are transduced with the virus containing the Cas plasmid. The cells are cultured for a time period sufficient to allow the CRISPR reaction to occur and the cells are then evaluated for CRISPR activity.

The provided method allows for evaluation of one or multiple variables in the CRISPR system. For example, as demonstrated herein, a single high-throughput competition assay was able to test three Cas9 variants across different PAM sites and different genome engineering tasks. Thus, in one embodiment, the method includes evaluation of multiple Cas proteins. Such proteins may be variants of the same Cas wild type protein, such as wild-type [WT] Cas9, Cas9-NG and xCas9, as shown herein. In one embodiment, one, two, three, four, five, six, seven, eight, nine, ten or more Cas variants are evaluated simultaneously. In one embodiment, the Cas proteins are Cas9 proteins.

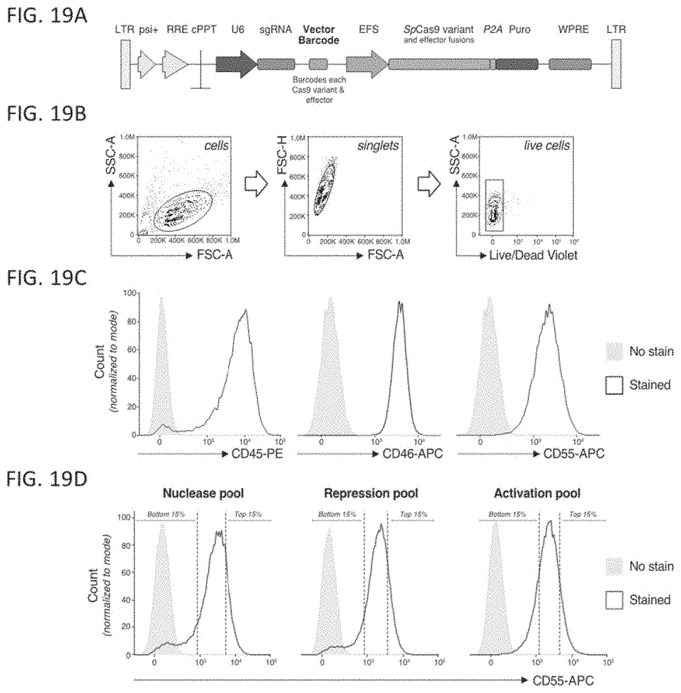

Plasmids are designed to express each Cas protein to be tested. For example, as described herein, human codon optimized Cas9 from lentiCRISPR v2 plasmid (Addgene 52961, Sanjana et al., 2014) as background for xCas9 and Cas9-NG mutations. xCas9 (also known as xCas3.7) mutations are as follows: A262T, R324L, S409I, E480K, E543D, M694I and E1219V (Hu et al., 2018). Cas9-NG mutations are: L1111R, D1135V, G1218R, E1219F, A1322R, R1335V, T1337R (Nishimasu et al., 2018). xCas9-NG mutations are: A262T, R324L, S409I, E480K, E543D, M694I, L1111R, D1135V, G1218R, E1219F, A1322R, R1335V and T1337R. For transcriptional modulation, Cas9 variants contained additional D10A and H840A mutations to make them catalytically inactive. KRAB domain was derived from pHAGE EF1α dCas9-KRAB (Addgene 50919, Kearns et al., 2014). VPR complex was derived from lenti-EF1a-dCas9-VPR-Puro (Addgene 99373, Ho et al., 2017) and modified to abolish BsmBI restriction sites. sgRNA scaffold was modified to improve its stability and Cas9 binding (F+E modification, Chen et al., 2013). Finally, we inserted a six-nucleotide barcode between the sgRNA scaffold and EFS promoter to act as an identifier for Cas9 variant and CRISPR modality (FIG. 19A). All cloning was performed by Gibson Assembly using recombinase-deficient NEB Stable cells (all from New England Biolabs). Cloned inserts were fully validated by Sanger sequencing (Eton Bioscience). All plasmids have been deposited on Addgene.

In another embodiment, the method includes evaluation of one or more CRISPR modalities or genetic perturbations. Such perturbations include nuclease activity, transcriptional activation (CRISPRa), and transcriptional repression (CRISPRi). For CRISPRa and CRISPRi, dCas9 variants may be used. In addition, Cas fusion proteins as described herein may be used. For example, for transcriptional activation (CRISPRa), nuclease-null versions of each Cas9 variant (D10A/H840A) may be fused to a transcriptional activation domain, such as VPR. VPR and other synergistic activators with multiple activation domains, such as SAM and SunTag, outperform single domain activators (Chavez et al., 2016). For transcriptional repression (CRISPR inhibition, CRISPRi), the nuclease-null Cas variants are fused to a transcriptional repression domain, such as the KRAB repressor domain (Kearns et al., 2014).

In another embodiment, the method includes evaluation of PAM specificity or flexibility of the Cas protein(s). In this embodiment, the sgRNA library is designed so that it targets sites spanning all possible three-nucleotide PAM combinations in the binding area of the selected gene. Such target sites may include coding exons (CDS) or a region within 3 kb of the transcription start site (TSS) of the selected gene. When evaluating CRISPR nuclease activity, the target sites may include CDS. When evaluating CRISRPi or CRISPRa activity, or both, the target sites may include TSS.

In one embodiment, the plasmid contains a barcode. The barcode is a short nucleotide sequence used to identify the particular Cas variant or specific modality being tested (functional domain present), or both. The barcode can be identified by sequencing, e.g., high-throughput sequencing. In one embodiment, the barcode is 3, 4, 5, 6, 7, 8, 9, 10, 11, 12, 13, 14, 15, 16, 17, 18, 19, or 20 nucleotides.

After culturing, the cells are evaluated for CRISPR activity, which may be done in various ways known to the person of skill in the art. The effect on targeted genes can be evaluated by FACS for cell surface proteins or by western blot or ELISA for any cellular protein. In addition, the effects can be evaluated at DNA level and/or mRNA transcript level by any form of DNA/transcription sequencing methods. It is desirable, in some embodiments, to select a gene that encodes a cell surface marker, which allows antibody staining and detection of expression via FACS or similar method. In another embodiment, high-throughput single-cell RNA sequencing is used to detect expression of the selected gene. See, e.g., Mimitou et al, Multiplexed detection of proteins, transcriptomes, clonotypes and CRISPR perturbations in single cells, Nature Methods, 16:409-12 (May 2019), which is incorporated herein by reference.

The following examples are illustrative only and are not intended to limit the present invention. The publication Legut, M. et al, High-Throughput Screens of PAM-Flexible Cas9 Variants for Gene Knockout and Transcriptional Modulation, Cell Reports, 30(9):2589-2868, ES, March 3, 2020 is incorporated herein by reference in its entirety.

### EXAMPLES

### Example 1

### A High-Throughput Competition Screen to Compare PAM-Flexible Cas9 Variants

To compare Cas9 variants across different PAM sites and different genome engineering tasks, we designed a high-throughput competition assay to test three Cas9 variants (WT Cas9, Cas9-NG, and xCas9) and three different genetic perturbations (nuclease, transcriptional activation, and transcriptional repression) at thousands of target sites in the human genome (FIG. 1A). For transcriptional activation (CRISPRa), we used nuclease-null versions of each Cas9 variant (D10A/H840A) fused to VPR proteins. VPR and other synergistic activators with multiple activation domains, such as SAM and SunTag, outperform single-domain activators (Chavez et al., 2016). For transcriptional repression (CRISPR inhibition [CRISPRi]), we tethered the nuclease-null variants to the KRAB repressor domain (Kearns et al., 2014). All Cas9 variant mutations were made on the same background using a human codon-optimized WT Cas9 from lentiCRISPRv2 (Sanjana et al., 2014) (FIG. 19A), and we noticed no differences in protein expression between Cas9 variants (FIG. 19B).

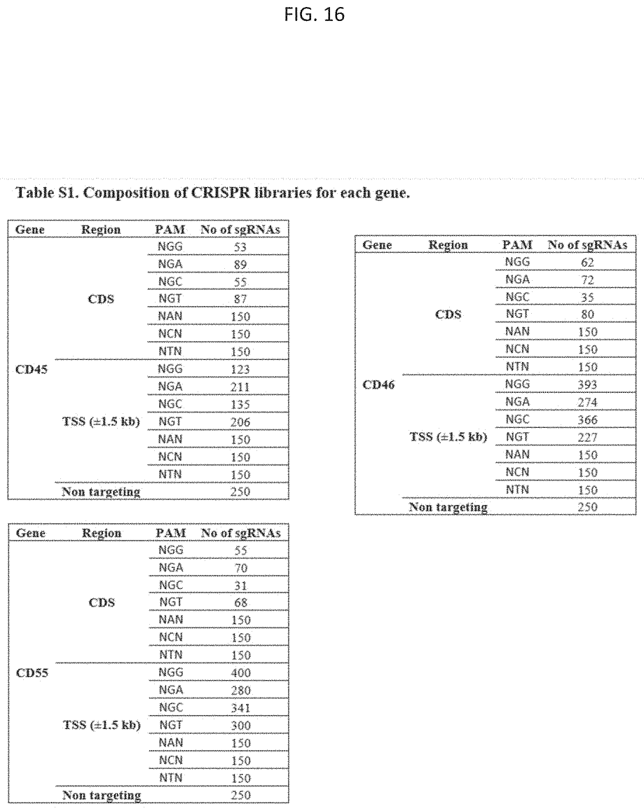

To build a sufficiently large dataset, we selected single-guide RNAs (sgRNAs) at thousands of target sites spanning all possible three-nucleotide PAM combinations. Specifically, we designed three sgRNA libraries targeting the genes CD45, CD46 and CD55, which encode cell surface markers that can be detected by antibody labeling, and are expressed in human K562 cells (FIG. 19B,C). For each gene-specific library, we selected sgRNAs that either target coding exons (CDS) or target within a 3 kb region flanking the transcription start site (TSS) (FIG. 1A). Combining TSS and CDS targeting sgRNAs in a single library enabled us to use the same library to test for CRISPR nuclease activity (assaying gene disruption) and transcriptional modulation via CRISPRi or CRISPRa. In the target regions (CDS and TSS), we selected all available NGN PAMs and equal numbers of NHN PAMs (FIG. 16). In total, we synthesized 6,713 sgRNAs targeting these three genes. Each gene-specific library also included 250 sgRNAs that are predicted to not target anywhere in the human genome as negative controls (Sanjana et al., 2014).

The libraries were cloned into a lentiviral plasmid containing a Cas9 variant (WT, Cas9-NG or xCas9) and a six-nucleotide barcode specific for the particular Cas9 variant and given modality (nuclease, repression or activation). This plasmid design allowed us to determine simultaneously the sgRNA and Cas9 effector (barcode) identities by high-throughput Illumina sequencing (FIG. 19A). Recently, several groups have reported lentiviral recombination between pseudodiploid viral RNAs as a function of distance within the viral RNA genome, which results in barcode swapping after transduction (Feldman et al., 2018; Hegde et al., 2018; Hill et al., 2018; Xie et al., 2018). To avoid these issues, we cloned and produced lentivirus separately for all 27 combinations of sgRNA libraries (CD45, CD46, CD55), Cas9 variants (WT, Cas9-NG or xCas9) and effector domains (nuclease, CRISPRi, CRISPRa). We separately transduced these libraries at a low multiplicity of infection into human K562 cells.

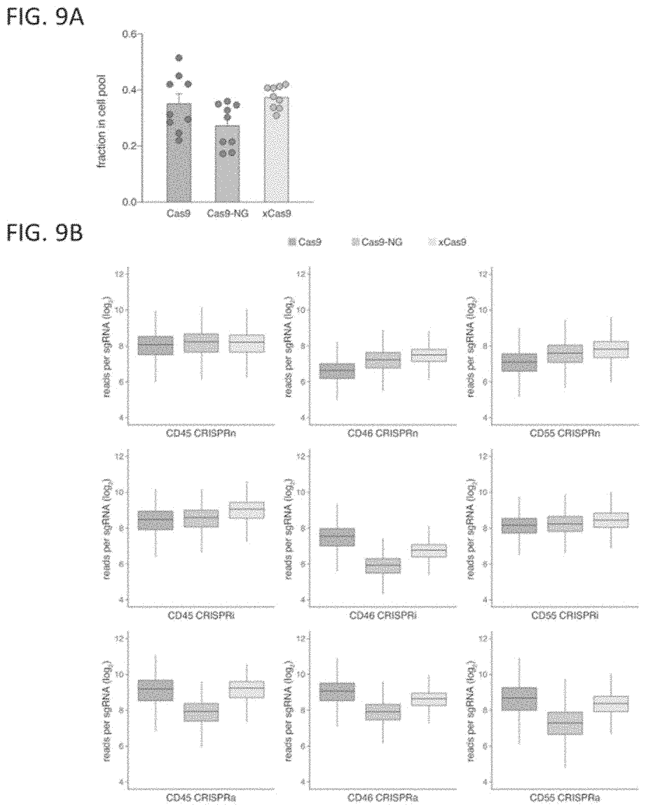

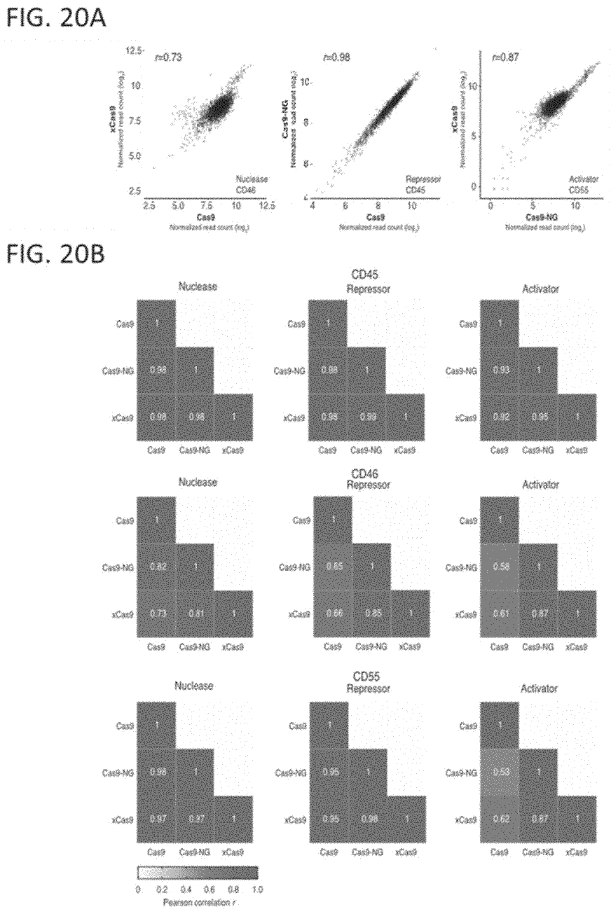

Following puromycin selection of transduced cells, we pooled together an equal number of cells transduced with different enzymes (WT, Cas9-NG or xCas9), performed antibody staining for each cell-surface protein, and sorted them by target expression via fluorescence-assisted cell sorting (FACS) (FIG. 19D). Pooling the cells just prior to antibody labelling and sorting allowed us to compare the efficiency of each enzyme in a direct competition-like assay, as well as to tightly control the ratios of cells transduced with each enzyme in the pre-sort input, to ensure no prior bias towards any Cas9 variant (FIG. 9). The relative frequency of every sgRNA-Cas9 variant pair from the top bin (highest expression) was then divided by its corresponding frequency in the bottom bin (lowest expression) to calculate the fold-change of sgRNAs associated with a particular PAM. In most cases, we found that the sgRNA distributions between Cas9 libraries in the mixed, pre-sort samples were tightly correlated (FIG. 20).

Cas9-NG Targets NGH PAMs with 2- to 4-Fold Lower Nuclease Activity than Cas9 at NGG PAMs

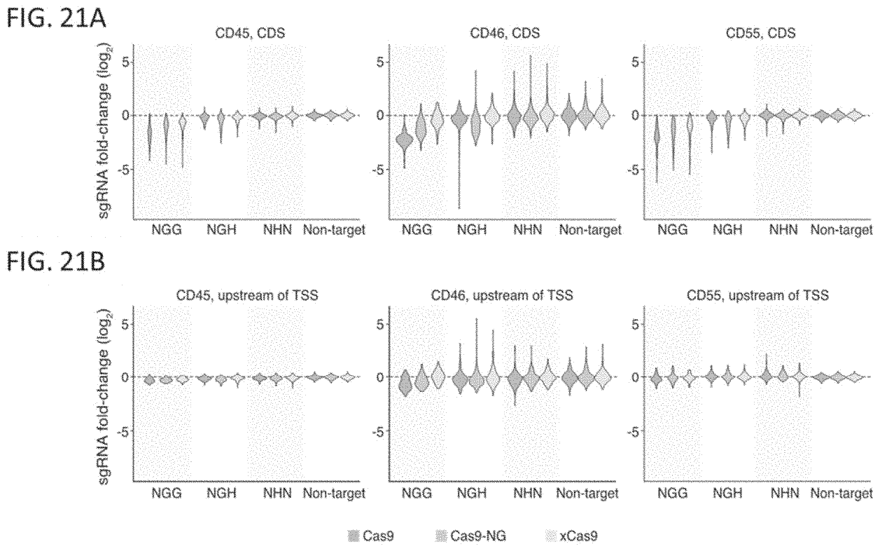

We first performed the CRISPR competition screens using catalytically-active nucleases and compared the fold-change of sgRNAs targeting coding exons (n=2,107 sgRNAs). Across all three cell-surface proteins, we observed the greatest fold-change for target sites with the canonical NGG PAM using the WT Cas9 enzyme (FIG. 1B; FIG. 21A shows each gene separately). Compared to WT Cas9, we found that the mean relative knock-out activity of Cas9-NG was 64% of WT and xCas9 was 43% of WT. For NGH PAMs, Cas9-NG provided the best overall knock-out (FIG. 1B). Unexpectedly, xCas9 was not significantly better than WT Cas9 at NGH PAMs. In contrast to CDS-targeting sgRNAs, sgRNAs targeting upstream noncoding regions for each of the three cell-surface proteins displayed only a minimal change in representation (FIG. 21B).

To further dissect Cas9 variant activity at specific PAMs and to discover potentially targetable non-NG PAMs, we next examined all possible nucleotide combinations at PAM positions 2 and 3 (FIG. 1C). While WT Cas9 showed the strongest activity at NGG PAMs, it was also capable of targeting endogenous genomic loci with all three NGH PAMs, albeit with greatly reduced activity. In addition to NGH PAMs, WT Cas9 showed significant recognition of NAG and NAA PAMs. Other groups have previously reported limited Cas9 nuclease activity in human cells at NAG PAMs, thus highlighting the sensitivity of our assay (Hsu et al., 2013; Zhang et al., 2015). Surprisingly, we found that xCas9 performed worse than WT Cas9 at all 3 NGH PAMs while PAM-flexible Cas9-NG was considerably more active than WT Cas9 or xCas9. Among NGH sites, Cas9-NG showed greatest activity at NGT PAMs and lowest activity at NGC PAMs, as reported previously (Nishimasu et al., 2018). In our screen, we also found that Cas9-NG was active at some non-NG PAMs, in particular at NAD (D=A, G or T) PAMs.

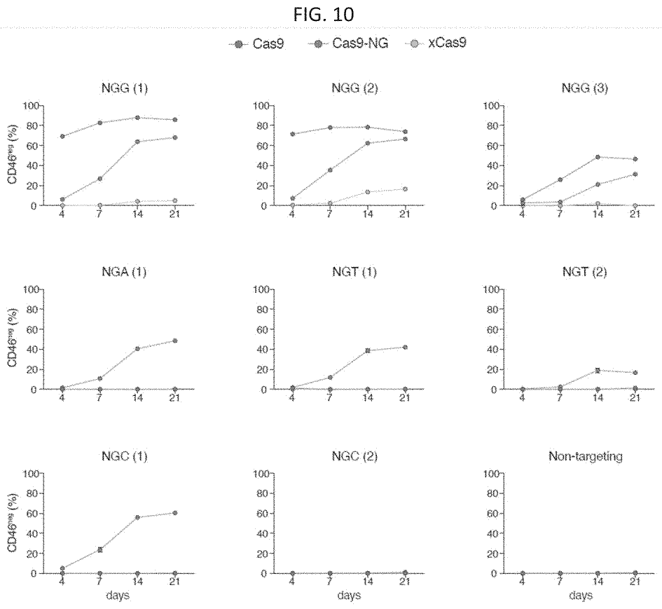

To further validate our pooled comparison, we targeted the CD46 gene in K562 cells with 18 individual sgRNAs at NGG and NGH PAMs using all 3 enzymes and quantified protein expression via FACS. To minimize bias due to sgRNA nucleotide composition, we designed sgRNAs targeting NGH PAMs to be shifted one nucleotide downstream from the corresponding NGG PAM-targeting sgRNAs. Following lentiviral transduction and selection, we measured the knockout efficiency by flow cytometry (FIG. 1D,E). We observed robust gene knockout induced by WT Cas9 and sgRNAs targeting NGG PAMs with 64% of cells having a CD46¹ phenotype. Cas9-NG at NGG PAMs induced full knockout at 46% efficiency of WT Cas9, followed by xCas9 at 7% of WT. At NGH PAMs, we could not detect any knockout above background induced by either WT Cas9 or xCas9; Cas9-NG activity at NGH PAMs was at 66% of its activity at corresponding NGG sites. Furthermore, xCas9 activity at NGG or NGH PAMs could not be rescued by increasing the editing time—even at day 21 post-transduction, knockout frequency with the best NGG sgRNA reached only 25% of knockout observed with Cas9-NG (FIG. 10).

Interestingly, we noticed a difference in knockout kinetics between wild-type Cas9 and Cas9-NG. While knockout efficiency of Cas9-NG (at both NGG and NGH PAM sites) sharply increased between days 4 and 14 post-transduction, wild-type Cas9 activity reached levels close to saturation already at day 4 (FIG. 10). Finally, both Cas9-NG and xCas9 showed high variability in knockout efficiency between different sgRNAs, ranging from no detectable activity up to a maximum of 17% (xCas9) or 70% (Cas9-NG) CD46^{neg} cells. This observation highlights the advantage of our approach: testing thousands of sgRNAs in parallel can reduce target site-specific bias by averaging over many target sites.

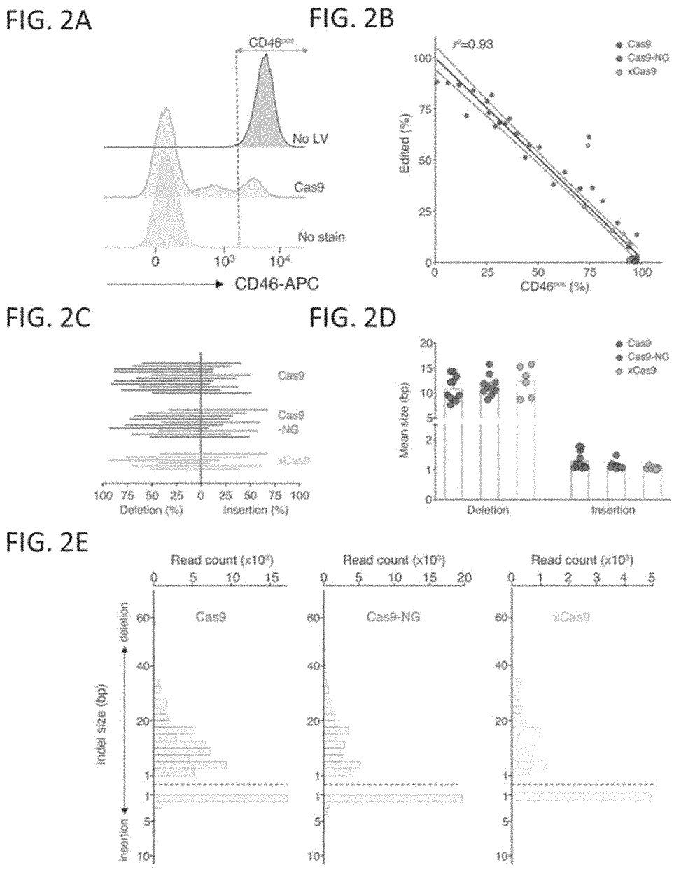

We also measured the editing efficiency at the DNA level by high-throughput amplicon sequencing and we observed that the frequency of alleles with insertions or deletions (indels) correlated well with protein expression from flow cytometry (r²=0.93, FIG. 2A,B). Furthermore, there was no significant difference between the three Cas9 variants with regard to their preferences for insertions or deletions or to the mean indel size among edited alleles (FIG. 2C-E).

Cas9-NG, but not xCas9 or WT Cas9, Efficiently Modulates Gene Expression at NGH PAMs

CRISPR nuclease activity is a two-step process: first, the Cas9-sgRNA complex binds the target DNA and second, it undergoes a conformational change which enables double-strand break formation (Nishimasu et al., 2018; Wu et al., 2014). In contrast, CRISPR transcriptional modulation only requires Cas9 sgRNA binding in the target region to enable recruitment of transcriptional repressors or activators. We hypothesized that xCas9, which showed suboptimal performance as a nuclease, might perform better in context of CRISPRi and CRISPRa because it was evolved via selection for DNA binding without cleavage. In the phage-based evolution and selection assay used to derive xCas9, nuclease-null Cas9 (dCas9) was fused to an E. coli RNA polymerase and targeted upstream of an essential gene for phage replication (Hu et al., 2018). In that study, xCas9 was shown to have, on average, a 12-fold increase in activity in human cells over WT Cas9 when fused to the VPR transcriptional activator (Hu et al., 2018). Given our previous results with xCas9 nuclease, we wanted to determine if dCas9 variants of the 3 enzymes fused to transcriptional activators and repressors would result in greater activity at NGH PAMs.

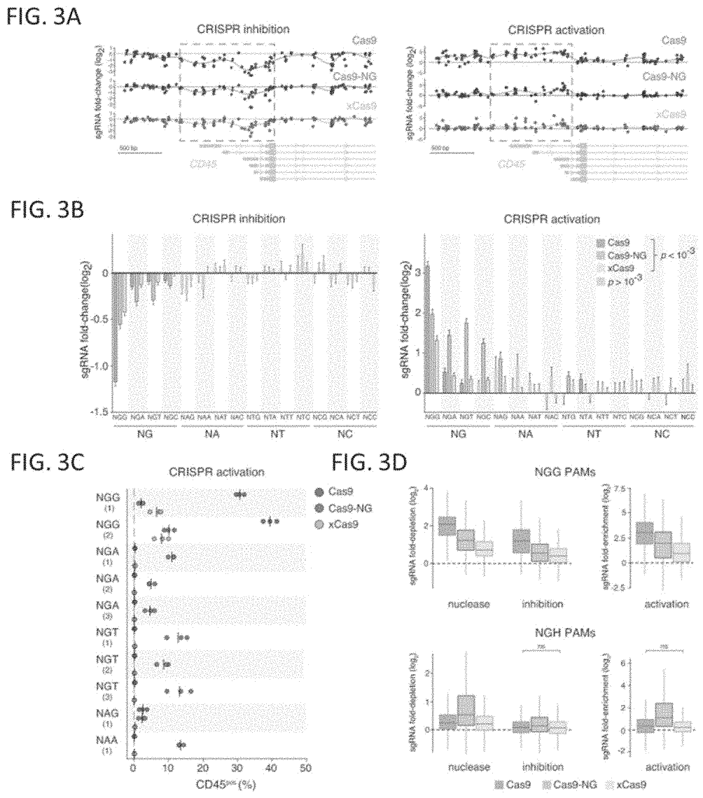

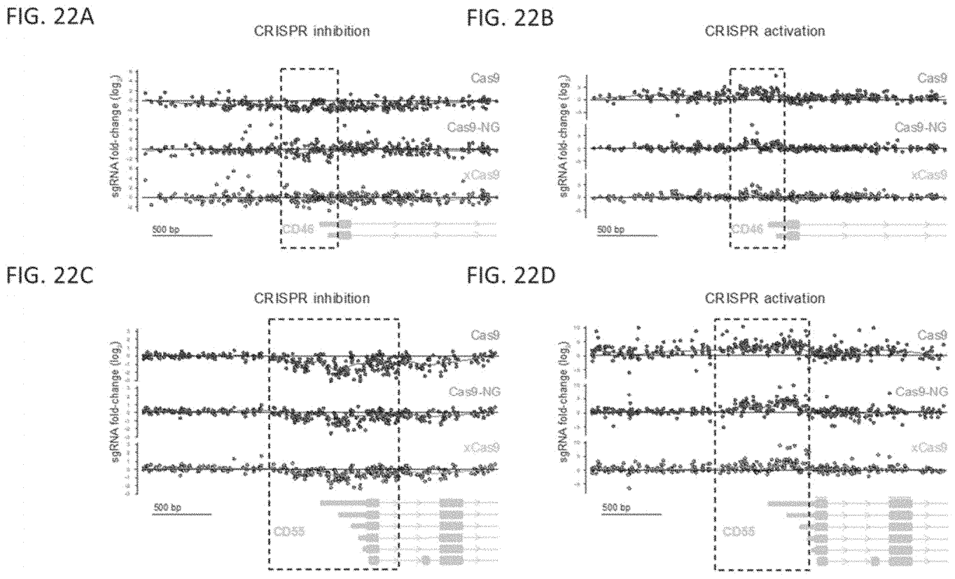

For this purpose, we first examined sgRNAs for all NGG PAMs tiling the 3 kb region surrounding the gene's primary TSS to identify the optimal target region for subsequent analysis and comparison across all PAMs. In general, we found that the optimal CRISPRi window was shifted downstream of the optimal CRISPRa window by ˜120 bp, possibly resulting from the interference of the bound Cas9 complex with the assembly of transcriptional machinery at the TSS (FIG. 3A, FIG. 22). Previously, Doench and colleagues reported that for CRISPR inhibition, the optimal targeting window is between +25 and +75 bp downstream of the TSS while for CRISPRa the optimal window lies between −150 and −75 bp upstream of the TSS (Sanson et al., 2018). We found similar windows for optimal CRISPRi and CRISPRa transcriptional modulation with peak CRISPRi inhibition downstream (3′) of peak CRISPRa activation. In addition, our screen data showed multiple peaks that aligned with particular transcript isoforms, suggesting that sgRNA positioning could preferentially activate or repress transcription from a particular TSS.

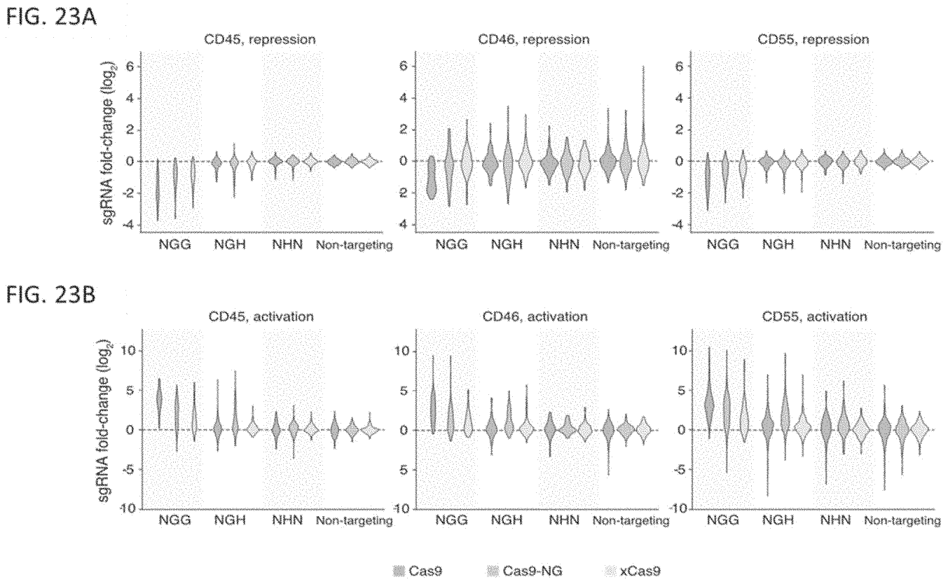

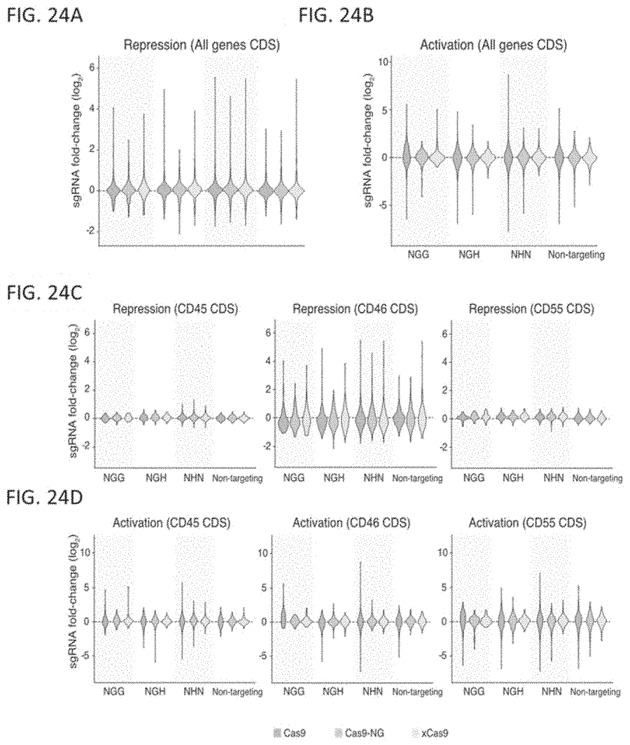

Overall, we observed that WT dCas9 produced the strongest effect on transcriptional modulation at NGG PAMs (FIG. 3B, FIG. 23). At NGH PAMs, dCas9-NG outperformed the other enzymes while dxCas9 had a similarly low activity to WT dCas9 at these PAMs, suggesting that xCas9 may not bind NGH PAMs as strongly as Cas9-NG. We also detected significant activity of dCas9-NG at unconventional NAD PAMs in context of CRISPR activation. This result is in agreement with our previous finding of Cas9-NG nuclease activity at NAD PAMs (FIG. 1C). As expected, there was no apparent difference between PAM sites or Cas9 variants when we looked at the fold-change of sgRNAs targeting CDS exons distant from the TSS (FIG. 24).

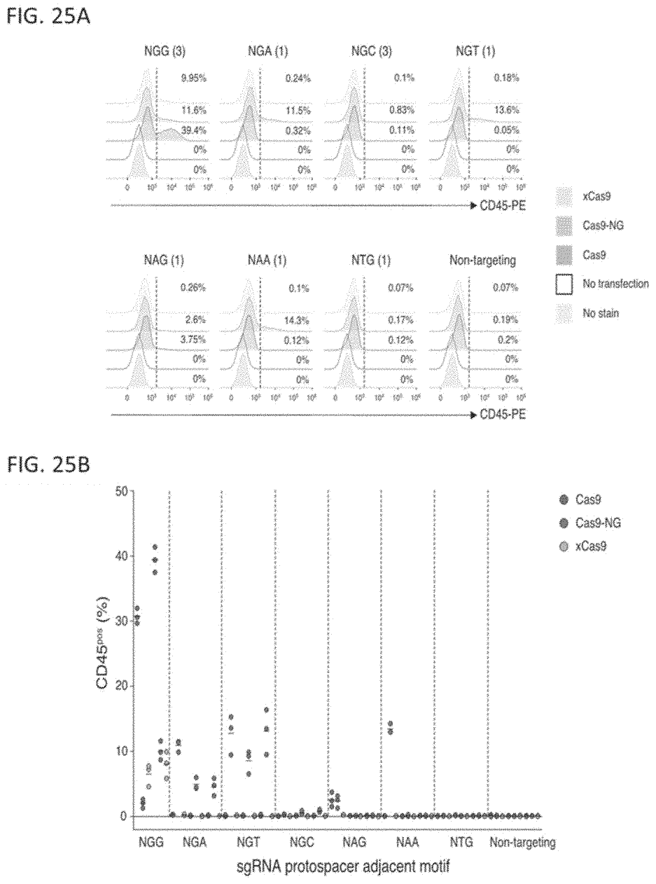

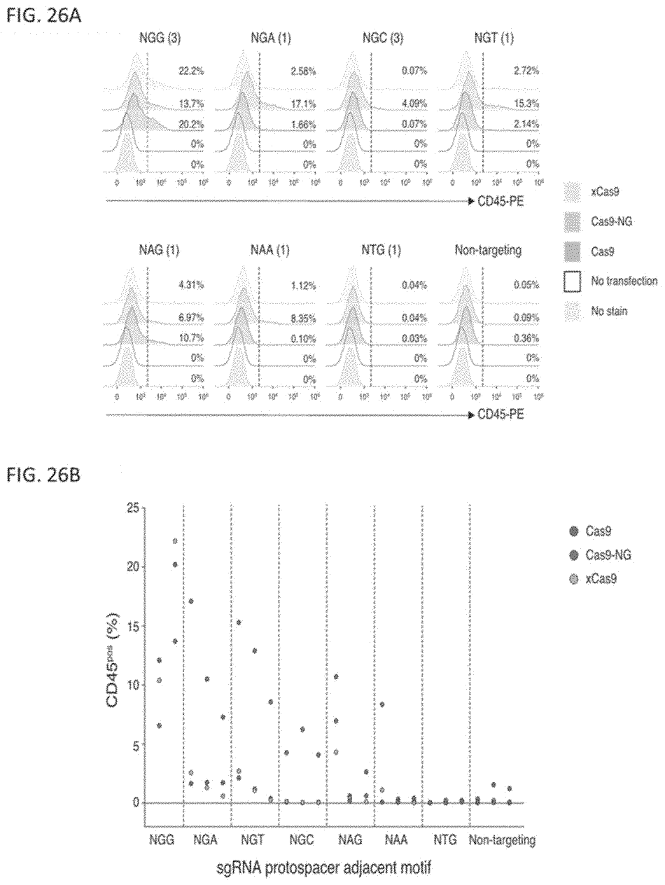

To further validate the pooled competition screen results, we targeted CD45 gene expression using 23 individual sgRNAs in two CD45^{neg} cell lines, A375 (FIG. 3C, FIG. 25) and HEK293T (FIG. 26), using CRISPRa. In addition to NGN PAMs, we also used unconventional NAD PAMs identified from our CRISPRn and CRISPRa screen analyses. WT Cas9 outperformed the PAM-flexible enzymes at the two NGG sites tested. For NGH PAMs, Cas9-NG demonstrated greater activity at NGT over NGA PAMs, in agreement with the pooled screen. We also detected Cas9-NG activity at one out of three NAG and one out of three NAA sites tested. Although xCas9 showed similar activity at NGG sites to Cas9-NG, there was no detectable CRISPRa-driven CD45 protein expression when targeting non-NGG sites with xCas9.

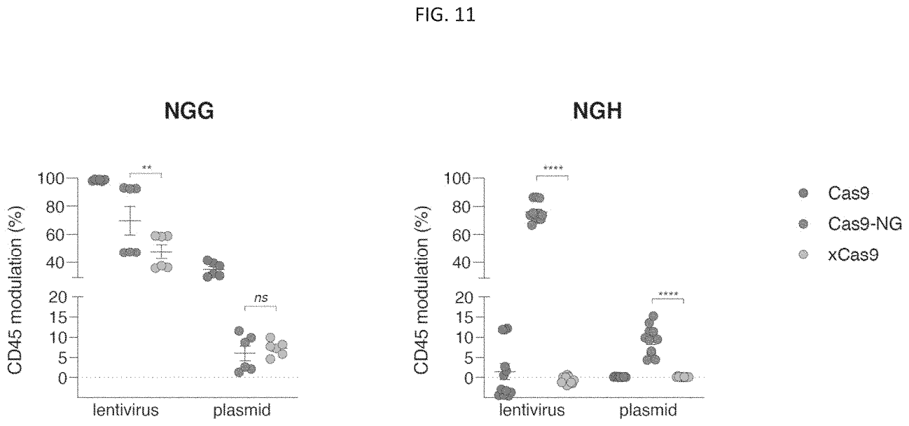

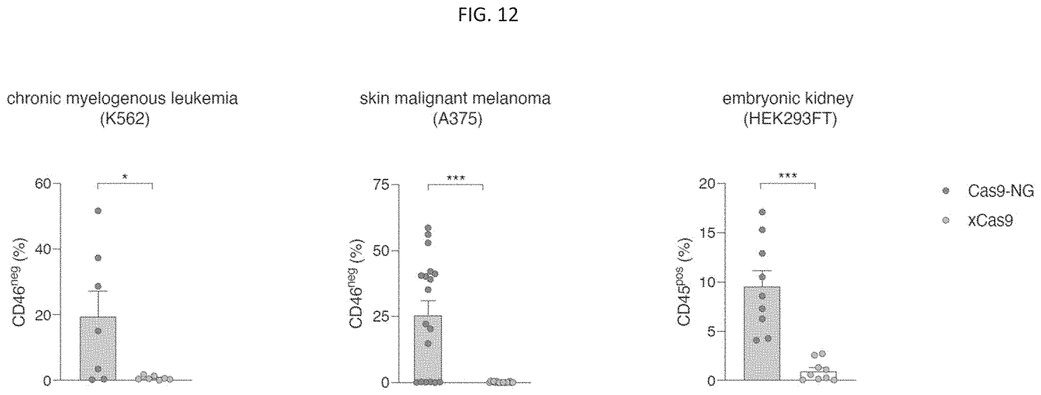

We next computed the relative activity of all three Cas9 enzymes at NGG and NGH PAMs, across all three modalities tested (nuclease, transcriptional activation, transcriptional repression), integrating data from nine separate CRISPR competition screens (FIG. 3D). At NGG PAMs, the strongest effector was WT Cas9 regardless of the modality, followed by Cas9-NG and then xCas9. At NGH PAMs, Cas9-NG showed significantly stronger activity than either WT Cas9 or xCas9. We found that xCas9 activity was not statistically different from WT Cas9 for transcriptional activation and repression at NGH PAMs; for nuclease activity, xCas9 was slightly, but significantly, weaker than WT Cas9. Overall, in three cell lines tested, Cas9-NG significantly outperformed xCas9 at NGH sites (FIG. 12). Similar results were obtained using both lentiviral transduction and plasmid transfection (FIG. 11).

### Example 2

### Introduction of Cas9-NG Mutations in xCas9 Partially Rescues Nuclease Activity and Increases Transcriptional Activation at NGH PAMs

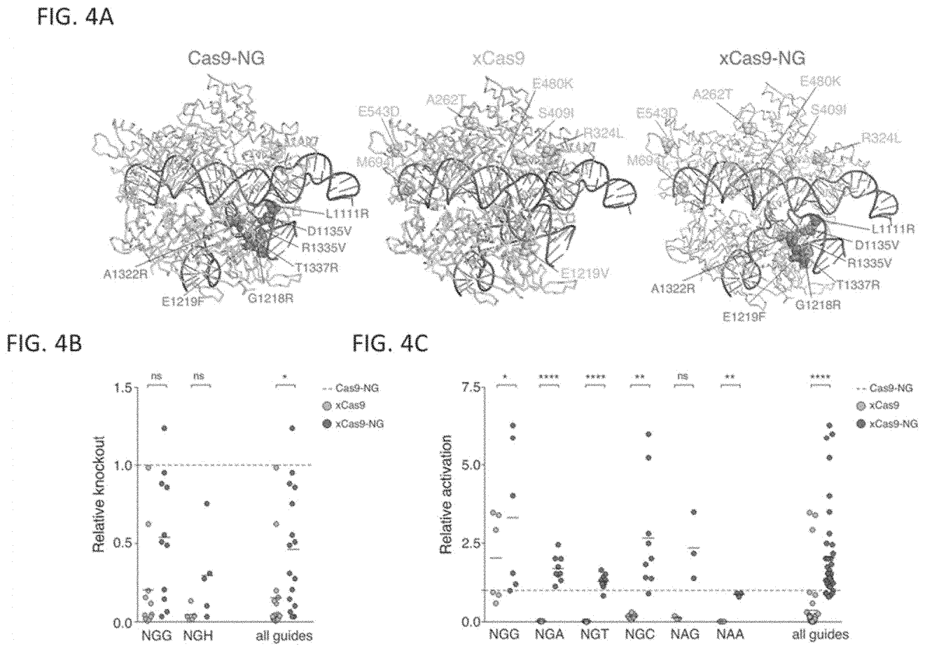

Our high-throughput CRISPR pooled competition screens and arrayed sgRNA validation data indicated that Cas9-NG is active for all modalities at NGN PAMs, albeit to a lesser extent than WT Cas9 at NGG sites. We also found that xCas9 had the poorest performance at virtually all PAMs and for all modalities. Due to this marked difference in Cas9-NG and xCas9 activity, we examined the position of the mutations in both Cas9 variants (FIG. 4A). The mutations in Cas9-NG cluster together in the PAM-interacting domain, as expected from structure-guided design. Conversely, xCas9 mutations, generated through directed evolution, are spread throughout the protein, with only one mutated residue (E1219) in common with Cas9-NG. Given their disparate positions in the protein, we wondered if it might be possible to rescue xCas9 activity using mutations from Cas9-NG. For this purpose, we created a new Cas9 variant that combines mutations from both xCas9 and Cas9-NG (with E1219F from Cas9-NG) and termed this novel variant xCas9-NG.

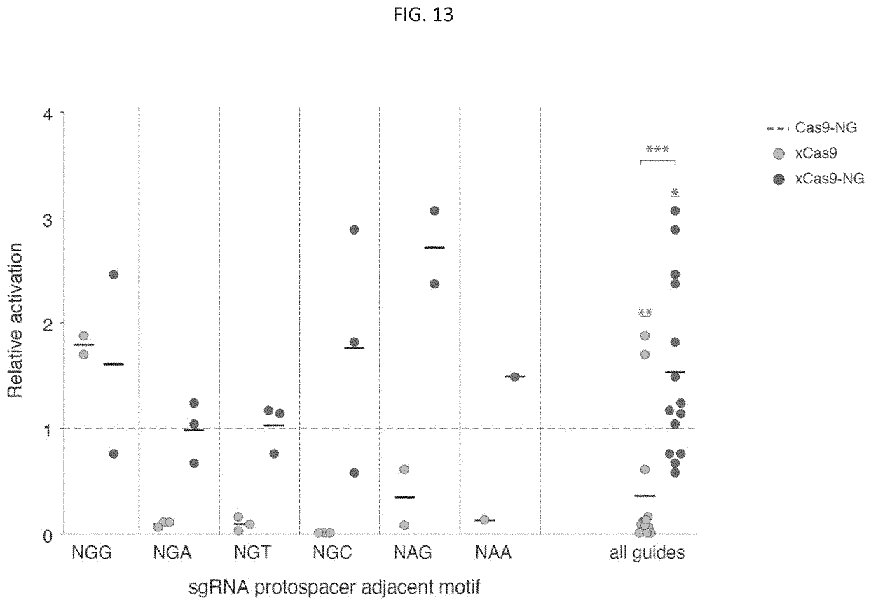

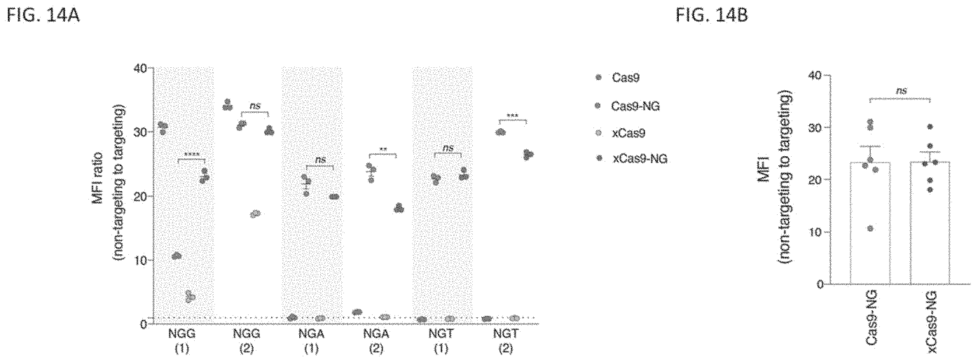

Using Cas9-NG as a baseline, we compared xCas9 and xCas9-NG nucleases using several sgRNAs to target CD46 in K562 cells at both NGG and NGH PAMs (FIG. 4B). For comparison, we normalized the effects of each sgRNA with either xCas9 or xCas9-NG to the same sgRNA with Cas9-NG. The ability of xCas9-NG to drive gene knockout was overall three times stronger than that of xCas9 but remained at ˜50% of Cas9-NG activity. For CRISPRa, the mean dxCas9-NG activation was on average two-fold greater than dCas9-NG and over five-fold greater than xCas9, across virtually all sgRNAs and for all NGN PAMs (FIG. 4C). In particular, dxCas9-NG had 2.7-fold higher activation than dCas9-NG at NGC PAMs, which is especially important given that Cas9-NG had very low activity at these PAMs. In an independent cell line (HEK293FT), we confirmed that xCas9-NG resulted in significantly greater transcriptional activation, albeit to a lesser extent than in A375 cell line, than either existing PAM-flexible Cas9 variant (FIG. 13). For CRISPR inhibition, xCas9-NG outperformed xCas9 with virtually every sgRNA tested, as well as outperformed Cas9-NG with one out of two NGG sgRNAs (FIG. 14A). Overall, when looking at all 6 NGN sites tested, xCas9-NG drove the same level of transcriptional repression as Cas9-NG (FIG. 14B). Thus, xCas9-NG appears to be a generally stronger transcriptional activator and an equal transcriptional repressor as Cas9-NG which may be possibly due to mechanistic differences between CRISPR activation and repression.

### Example 3

### Discussion

Taken together, we performed nine independent CRISPR competition screens, spanning three endogenously expressed human genes and three CRISPR modalities, to assess the efficacy of recently-described PAM-flexible Cas9 variants at different PAM sites. These are the first pooled CRISPR screens using xCas9 or Cas9-NG, testing thousands of endogenous genomic loci in a massively-parallel manner. By combining cells transduced with all three Cas9 variants prior to FACS, we were able to perform a pooled comparison where each variant competes against other variants. This high-throughput CRISPR competition screen provides a general method of assessing relative efficacies of PAM-flexible Cas9 variants and provides a far richer dataset than previous work with only a few target sites (Hu et al., 2018; Nishimasu et al., 2018). While this screen was not designed to discover sequence features determining the on-target efficiency of PAM-flexible Cas9 enzymes, that could be achieved by scaling up the number of assayed sgRNAs.

We showed that the mutations that increase PAM flexibility of Cas9 lead to decreased activity of these enzymes at NGG target sites. This observation applies to both catalytically active and inactive Cas9 variants. When comparing Cas9 variants at target sites with NGH PAMs, we were surprised to discover that while Cas9-NG maintains a similar level of activity as for target sites with NGG PAMs, the activity of xCas9 was profoundly diminished. In fact, at target sites with NGH PAMs, xCas9 did not perform better than wild-type Cas9 across all modalities tested (nuclease, activation, and inhibition). The discrepancies between the results reported in this study and in the original xCas9 publication could potentially stem from differences in accessibility of the target sites, thus highlighting the need to test endogenous loci for meaningful comparisons. Recent studies in plants (Ge et al., 2019; Hua et al., 2019; Negishi et al., 2019; Wang et al., 2019; Zhong et al., 2019) have shown that the overall efficiency of indel formation and base editing at non-NGG sites is much higher for Cas9-NG than for xCas9, supporting our findings in the mammalian context. Furthermore, David Liu and colleagues recently demonstrated that Cas9-NG base editors outperform xCas9 base editors at target sites with NGH PAMs and observed very low or no editing at the vast majority of loci tested when using xCas9 (Huang et al., 2019).

Structural studies have shown that the mechanisms behind relaxed PAM recognition by xCas9 and Cas9-NG are considerably different. In case of Cas9-NG, NG PAM recognition is enabled by mutating both the R1335 residue interacting with the third nucleobase of the PAM (dG3), and E1219, which stabilizes R1335. The remaining five mutations are introduced to enhance Cas9-NG binding to the now smaller, two-nucleobase PAM (Nishimasu et al., 2018). Conversely, in xCas9 the R1335-dG3 interaction is disrupted indirectly, by abrogating the E1219-R1335 interaction and allowing R1335 to adopt multiple conformations (Guo et al., 2019). The remaining xCas9 mutations are located in the recognition (REC) lobes and result in the conformational change of Cas9 binding to DNA.

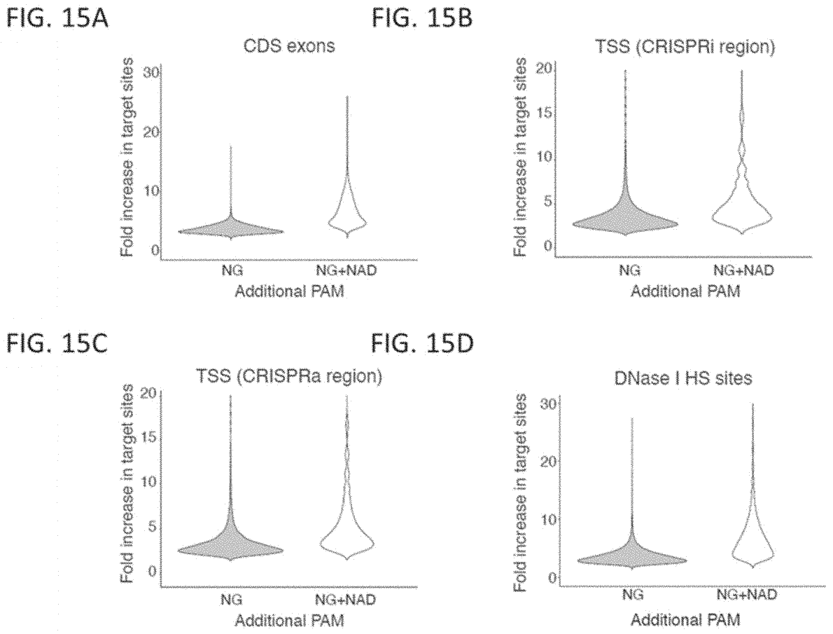

Given these differences, we investigated how the change of REC lobes conformation (xCas9 mutations) would affect the editing activity of the enzyme when combined with enhanced binding to the two-nucleobase PAM (Cas9-NG mutations). This new Cas9 variant, termed xCas9-NG, showed improved nuclease activity compared to xCas9, presumably due to stronger interactions with the PAM, although it did not fully rescue nuclease activity to the Cas9-NG level. In contrast, we also found that xCas9-NG was superior to both xCas9 and Cas9-NG for transcriptional modulation, possibly indicating that a more relaxed REC lobe interaction with target DNA allows for easier access of the recruited transcriptional machinery. Over the entire human exome and functional non-coding regions, the relaxed PAM constraints of xCas9-NG enable a significantly larger target space (FIG. 15A-D), especially when considering the additional NAD PAMs found in our screens.

As none of the three PAM flexible Cas9 mutants were capable of matching the efficacy of wild-type Cas9 at NGG PAM sites, relaxing PAM interactions through these mutations likely incurs a fitness cost in enzyme performance. New strategies are needed for designing efficient, PAM-flexible (or perhaps even PAM-independent) Cas9 enzymes. The CRISPR competition screen presented here provides a robust and scalable platform for future benchmarking of different genome editing enzymes prior to their implementation in research, clinical or industrial applications.

### Example 4

### Materials and Methods

### Cell Culture

K562 and A375 cell lines were obtained from ATCC. HEK 293FT cells were obtained from Thermo Scientific. K562 cells were cultured in Iscove's Modified Dulbecco's Medium (IMDM); A375 and HEK293FT cells were cultured in Dulbecco's Modified Eagle Medium (DMEM). All media were from Caisson Labs. Media were supplemented with 10% Serum Plus II Medium Supplement (Sigma-Aldrich). Cells were regularly passaged and tested for presence of mycoplasma contamination (MycoAlert Plus Mycoplasma Detection Kit, Lonza).

### Plasmid Design

In order to enable a meaningful comparison between different Cas9 variants, we used the human codon optimized Cas9 from lentiCRISPR v2 plasmid (Addgene 52961, Sanjana et al., 2014) as background for xCas9 and Cas9-NG mutations. xCas9 (also known as xCas3.7) mutations are as follows: A262T, R324L, S409I, E480K, E543D, M694I and E1219V (Hu et al., 2018). Cas9-NG mutations are: L1111R, D1135V, G1218R, E1219F, A1322R, R1335V, T1337R (Nishimasu et al., 2018). xCas9-NG mutations are: A262T, R324L, S409I, E480K, E543D, M694I, L1111R, D1135V, G1218R, E1219F, A1322R, R1335V and T1337R. For transcriptional modulation, Cas9 variants contained additional DMA and H840A mutations to make them catalytically inactive. KRAB domain was derived from pHAGE EF1α dCas9-KRAB (Addgene 50919, Kearns et al., 2014). VPR complex was derived from lenti-EF1a-dCas9-VPR-Puro (Addgene 99373, Ho et al., 2017) and modified to abolish BsmBI restriction sites. sgRNA scaffold was modified to improve its stability and Cas9 binding (F+E modification, Chen et al., 2013). Finally, we inserted a six-nucleotide barcode between the sgRNA scaffold and EFS promoter to act as an identifier for Cas9 variant and CRISPR modality (FIG. 19A). All cloning was performed by Gibson Assembly using recombinase-deficient NEB Stable cells (all from New England Biolabs). Cloned inserts were fully validated by Sanger sequencing (Eton Bioscience). All plasmids have been deposited on Addgene.

Lentiviral sgRNA Library Design nd Cloning

The sgRNAs targeting the 3 kb region surrounding the TSS and constitutive protein-coding exons were chosen to include all possible 20-mer sequences upstream of an NG PAM sequence, and equal numbers of 20-mer sequences upstream of NH PAM sequences. Primary TSS and exon annotations were obtained from the UCSC Genome Browser based on the hg38 genome assembly. We also included 250 non-targeting sgRNAs from the GeCKO v2 library (Sanjana et al., 2014) as a negative control in each library. FIG. 16 specifies the number of sgRNAs per category. The sgRNA library was synthesized as an oligo pool of 103 nt oligos (Twist Bioscience) and cloned using Gibson Assembly (New England Biolabs) into Esp3I-digested (ThermoScientific/Fermentas) lentiviral transfer plasmids containing Cas9 effectors. The cloned libraries were individually amplified by electroporation into Endura ElectroCompetent cells (Lucigen). Using dilution plates for colony counting, we verified that all libraries were cloned with >1,000 x library coverage. Plasmids with cloned libraries were sequenced to confirm representation (MiSeq).

### Production of Lentivirus and Transduction

Lentivirus was produced by polyethylenimine linear MW 25000 (Polysciences 23966) transfection of HEK 293FT cells with the transfer plasmid containing a barcoded Cas9 effector and sgRNA library, packaging plasmid psPAX2 (Addgene 12260) and envelope plasmid pMD2.G (Addgene 12259). After 72 h post-transfection, cell media containing lentiviral particles was harvested and filtered through 0.45 μm filter Steriflip-HV (Millipore SE1M003M00). Each sgRNA library and Cas9 effector combination was transduced into K562 cells individually, to avoid barcode swapping, and thus Cas9 misidentification, during lentiviral integration (Xie et al., 2018). In total we produced 27 individual lentiviral libraries and transduced them into separately into K562 cells. The transduction was performed at a multiplicity of infection (MOI) ˜0.4 to minimize the fraction of cells with multiple sgRNAs. We maintained 1,000× coverage of each sgRNA library. Transduced cells were selected with 1 μg ml⁻¹ puromycin for at least 7 days after transduction. During the course of the screen the cells were maintained at numbers ensuring >1,000× representation of the library. Transduced cells were maintained as 27 separate cell cultures for 14 days. At day 14 post-transduction, cells transduced with the sgRNA library targeting the same gene and the same CRISPR modality (but different Cas9 variants) were combined in equal numbers, resulting in 9 separate cell pools for screening, and then analyzed and sorted via FACS. All cell counting was done using a Cellometer Auto T4 counter (Nexcelom).

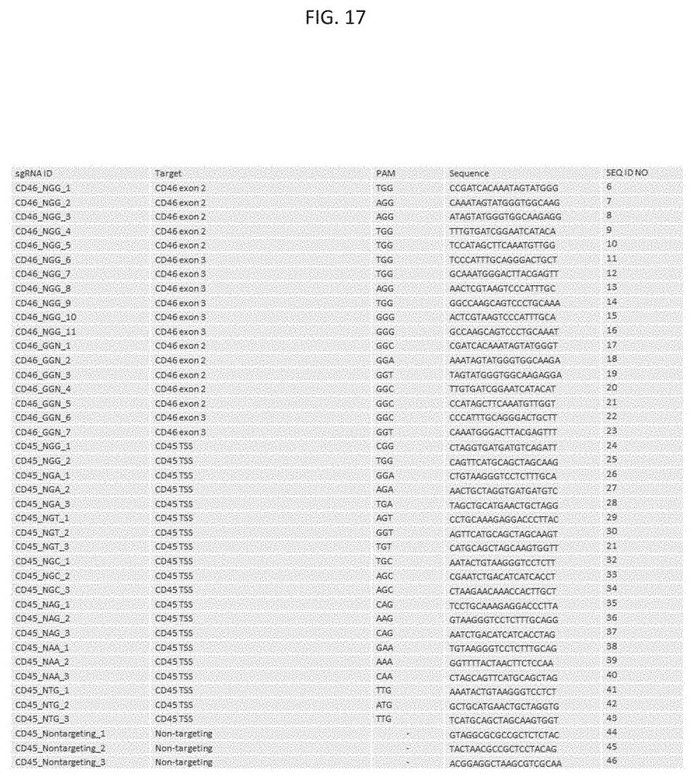

For arrayed CD46 knockout validation in K562 and A375 cells, sgRNAs targeting exons 2 and 3 of CD46 gene were designed in benchling software as 20-mers upstream of an NGG PAM, or by shifting +1 bp upstream, as 20-mers upstream of an NGH PAM (FIG. 17). The individual sgRNAs were cloned into lentiviral transfer plasmids encoding Cas9 variants and transduced into K562/A375 cells at MOI ˜0.5. K562 cells were assessed for CD46 knockout by flow cytometry on day 14 after transduction. At this timepoint an aliquot of cells was also collected for genomic DNA (gDNA) extraction. A375 cells were assessed for CD46 knockout by flow cytometry on days 4, 7, 14 and 21 after transduction.

For arrayed CRISPR inhibition validation, we selected guide RNAs from sequences included in the screen library (NG PAMs) or designed to target within close proximity to NG PAM sgRNAs (NH PAM). The sequences of sgRNAs are listed in FIG. 17. The individual sgRNAs were cloned into lentiviral transfer plasmids encoding Cas9 variants and transduced into K562 cells at MOI ˜0.5. K562 cells were assessed for CD45 knockdown by flow cytometry on day 14 after transduction.

### Transfection

For arrayed CRISPR activation validation, sgRNA-specifying oligos were either obtained from sgRNA sequences included in the screen library (NG PAMs) or designed to target within close proximity to NG PAM sgRNAs (NH PAM). The sequences of sgRNAs are listed in FIG. 17. The individual sgRNAs were cloned into a sgRNA-only plasmid with the F+E scaffold modification (Chen et al., 2013) and co-transfected with plasmids containing Cas9 effectors into A375 or HEK 293FT cells using Lipofectamine 2000 (ThermoFisher 11668019). The transfected cells were selected with 2 μg ml⁻¹ puromycin for 72 h. At day 4 post-transfection, the cells were assessed for CD45 expression by flow cytometry.

### Protein Expression

HEK293FT cells were transiently transfected with equal amounts of Cas9 variants expression vectors. At 24 hours post-transfection, the cells were collected, lysed with THE buffer (10 mM Tris-HCl, pH 7.4, 150 mM NaCl, 1 mM EDTA, 1% Nonidet P-40) supplemented with protease inhibitor cocktail (Bimake B14001) for 1 hour on ice. Cells lysates were spun for 10 min at 10,000 g, and supernatants were used to determine the protein concentration for each sample using the BCA assay (ThermoFisher 23227). Equal amounts of whole cell lysates (20 μg protein per sample) were denatured in Tris-Glycine SDS Sample buffer (ThermoFisher LC2676), and loaded on a Novex 4-20% Tris-Glycine gel (ThermoFisher XP04205BOX). PageRuler pre-stained protein ladder (ThermoFisher 26616) was used to determine the protein size. The gel was run in 1× Tris-Glycine-SDS buffer (IBI Scientific IBI01160) for 20 min at 80V, and then for additional 100 min at 120V. Proteins were transferred on a nitrocellulose membrane (BioRad 1620112) in presence of prechilled 1× Tris-Glycine transfer buffer (FisherSci LC3675) supplemented with 20% methanol for 100 min at 100V. Immunoblots were blocked with 5% skim milk dissolved in 1× PBS+1% Tween 20 (PBST), washed well with PBST and incubated overnight at 4° C. separately with the following primary antibodies: mouse anti-2A peptide, clone 3H4 (1 μg/mL, Millipore MABS2005); rabbit anti-GAPDH 14C10 (0.1 μg/mL, Cell Signaling 2118S). Following the primary antibody, the blots were incubated with IRDye 680RD donkey anti-rabbit (0.2 μg/mL, LI-COR 926-68073) or with IRDye 800CW donkey anti-mouse (0.2 μg/mL, LI-COR 926-32212). The blots were imaged using Odyssey CLx (LI-COR). Band intensity quantification was performed using ImageJ version 1.51.

### Flow Cytometry and FACS

For CRISPR library sorting, >10⁸ cells were taken for antibody staining (˜10,000×library representation). We set aside 10⁷ cells for the pre-sort control (˜1,000×coverage). After harvesting the cells and removing leftover medium by washing with PBS, the cells were stained for 5 minutes at room temperature with LIVE/DEAD Fixable Violet Dead Stain Kit (ThermoFisher L34864). Subsequently, the cells were stained with antibodies for 20 minutes on ice. The following antibodies were used: CD45-PE (clone 2D1), CD46-APC (clone TRA-2-10) or CD55-APC (clone JS11). All antibodies were purchased pre-conjugated from BioLegend. Cells were washed with PBS to remove unbound antibodies prior to sorting. Cell acquisition and sorting was performed using a Sony SH800S cell sorter.

Sequential gating was performed as follows: 1) exclusion of debris based on forward and side scatter cell parameters, 2) doublet exclusion, and 3) dead cell exclusion (FIG. 19B). The sorting gates were set based on the expression level of the target protein in sgRNA library-transduced cells (top and bottom 15% of expression, FIG. 19D). Typically, we achieved >500×library coverage within each sorted population.

### Pooled Crispr Competition Assay Sequencing

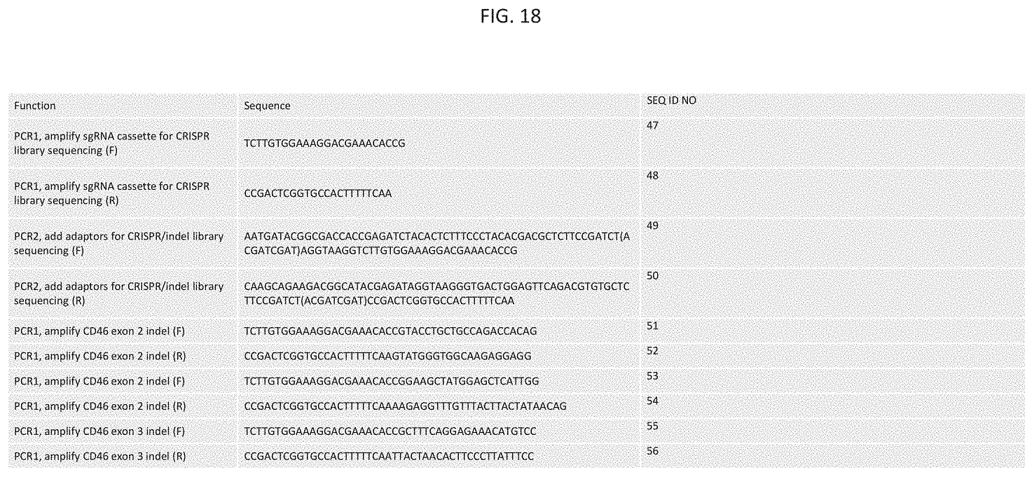

The sgRNA library preparation was performed as described before (Shalem et al., 2014). Briefly, gDNA was extracted using GeneJET DNA Purification Kit (Thermo Fisher Scientific). All of the extracted gDNA was then used in the first PCR reaction, in multiple reactions not exceeding 10 μg gDNA per 100 uL PCR reaction. Samples were then subjected to a second PCR to add sequencing adaptors and to barcode the samples. All PCR primers are listed in FIG. 18. PCR products were run on a 2% agarose gel and the correct size band was extracted. PCR products from different samples were then pooled together in equimolar ratios. Sequencing was performed on the NextSeq 500 instrument using the MidOutput Mode v2 with 75 bp paired-end reads (Illumina).

### Pooled CRISPR Competition Assay Data Analysis

The sgRNA sequences present in the sorted samples (read 1) as well as their corresponding barcodes indicating the Cas9 variant and CRISPR modality (read 2) were enumerated. sgRNA sequences were mapped to the reference sgRNA library with one mismatch allowed (bowtie -v 1 -m 1). Read numbers were normalized to the total number of reads per sample (with a pseudocount added to all sgRNAs) and loge-transformed. The median of non-targeting sgRNAs was calculated for each of the three Cas9 variants present in a sample. The median of non-targeting (NT) sgRNAs associated with each Cas9 was then used to normalize the sgRNA read counts associated with that Cas9. Finally, the fold-change of each NT-normalized sgRNA-Cas9 pair in top 15% bin was calculated over the NT-normalized sgRNA-Cas9 pair in the bottom 15% bin. Statistical significance was determined by two-sided Student's t-test with Bonferroni correction (RStudio). For CRISPRi and CRISPRa screens, we needed to determine optimal windows around the TSS to pick the sgRNAs for subsequent analyses (i.e. to compare Cas9 variants across NGN PAMs, and to identify new functional NHN PAMs). Windows were selected to capture the peak region identified from the LOESS fit for all three enzymes, using only the NGG sgRNAs for strongest signal. The following parameters were chosen for LOESS fitting using the Gviz package (Hahne and Ivanek, 2016) in RStudio: span=0.2, evaluation=500, degree=10.

### Nuclease Indel Sequencing

For validation of arrayed CD46 knockout, genomic DNA was isolated using QuickExtract DNA Extraction Solution (Epicentre). Two sets of PCR primers were designed: first set was flanking the exons to be amplified and contained handles for the second PCR. The primers for the second PCR were handle-specific, and added Illumina sequencing adaptors and indexes (FIG. 18). PCR products of the correct size were extracted following agarose gel electrophoresis, combined in equimolar amounts and sequenced on the NextSeq 500 instrument using the MidOutput Mode v2 with 150 bp single-end reads (Illumina).

### Data Analysis

Illumina single-end reads for CD46 genomic amplicons were analyzed using CRISPResso2 software (Clement et al., 2019) to quantify the fraction of reads containing editing at expected sites, and to determine the editing outcome in terms of indel type and size. Flow cytometry data was analyzed using FlowJo software. Visualization of Cas9 protein structures was performed in PyMOL software (PDB IDs: 4un3; 6ai6). All other data analysis was performed in GraphPad Prism 8 and RStudio. All correlation coefficients (r) and coefficients of determination (r²) are Pearson's correlation. DNase I hypersensitivity (HS) sites in the K562 cell line were downloaded from ENCODE DNase Uniformly Processed Peaks from UCSC based on hg19 genome build.

### Data Representation

In all boxplots, boxes indicate the median and interquartile ranges, with whiskers indicating either 1.5 times the interquartile range, or the most extreme data point outside the 1.5-fold interquartile. All transfection experiments show the mean of three replicate experiments, with error bars representing the standard error of mean.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

### REFERENCES

1. Anders, C., Niewoehner, O., Duerst, A., and Jinek, M. (2014). Structural basis of PAM-dependent target DNA recognition by the Cas9 endonuclease. Nature 513, 569-573.

2. Chavez, A., Tuttle, M., Pruitt, B. W., Ewen-Campen, B., Chari, R., Ter-Ovanesyan, D., Haque, S. J., Cecchi, R. J., Kowal, E. J. K., Buchthal, J., et al. (2016). Comparison of Cas9 activators in multiple species. Nature Methods 13, 563-567.

3. Chen, B., Gilbert, L. A., Cimini, B. A., Schnitzbauer, J., Zhang, W., Li, G.-W., Park, J., Blackburn, E. H., Weissman, J. S., Qi, L. S., et al. (2013). Dynamic Imaging of Genomic Loci in Living Human Cells by an Optimized CRISPR/Cas System. Cell 155, 1479-1491.

4. Clement, K., Rees, H., Canver, M. C., Gehrke, J. M., Farouni, R., Hsu, J. Y., Cole, M. A., Liu, D. R., Joung, J. K., Bauer, D. E., et al. (2019). CRISPResso2 provides accurate and rapid genome editing sequence analysis. Nature Biotechnology 37, 224-226.

5. Feldman, D., Singh, A., Garrity, A. J., and Blainey, P. C. (2018). Lentiviral co-packaging mitigates the effects of intermolecular recombination and multiple integrations in pooled genetic screens. BioRxiv.

6. Findlay, G. M., Boyle, E. A., Hause, R. J., Klein, J. C., and Shendure, J. (2014). Saturation editing of genomic regions by multiplex homology-directed repair. Nature 513, 120-123.

7. Ge, Z., Zheng, L., Zhao, Y., Jiang, J., Zhang, E. J., Liu, T., Gu, H., and Qu, L. (2019). Engineered xCas9 and SpCas9-NG variants broaden PAM recognition sites to generate mutations in Arabidopsis plants. Plant Biotechnology Journal.

8. Guo, M., Ren, K., Zhu, Y., Tang, Z., Wang, Y., Zhang, B., and Huang, Z. (2019). Structural insights into a high fidelity variant of SpCas9. Cell Research 29, 183-192.

9. Hahne, F., and Ivanek, R. (2016). Visualizing Genomic Data Using Gviz and Bioconductor. In Statistical Genomics, E. Mahe, and S. Davis, eds. (New York, NY: Springer New York), pp. 335-351.

10. Hegde, M., Strand, C., Hanna, R. E., and Doench, J. G. (2018). Uncoupling of sgRNAs from their associated barcodes during PCR amplification of combinatorial CRISPR screens. PLoS ONE 13, e0197547.

11. Hill, A. J., McFaline-Figueroa, J. L., Starita, L. M., Gasperini, M. J., Matreyek, K. A., Packer, J., Jackson, D., Shendure, J., and Trapnell, C. (2018). On the design of CRISPR-based single-cell molecular screens. Nature Methods 15, 271-274.

12. Ho, S.-M., Hartley, B. J., Flaherty, E., Rajarajan, P., Abdelaal, R., Obiorah, I., Barretto, N., Muhammad, H., Phatnani, H. P., Akbarian, S., et al. (2017). Evaluating Synthetic Activation and Repression of

13. Neuropsychiatric-Related Genes in hiPSC-Derived NPCs, Neurons, and Astrocytes. Stem Cell Reports 9, 615-628.

14. Hsu, P. D., Scott, D. A., Weinstein, J. A., Ran, F. A., Konermann, S., Agarwala, V., Li, Y., Fine, E. J., Wu, X., Shalem, O., et al. (2013). DNA targeting specificity of RNA-guided Cas9 nucleases. Nature Biotechnology 31,827-832.

15. Hu, J. H., Miller, S. M., Geurts, M. H., Tang, W., Chen, L., Sun, N., Zeina, C. M., Gao, X., Rees, H. A., Lin, Z., et al. (2018). Evolved Cas9 variants with broad PAM compatibility and high DNA specificity. Nature 556,57-63.

16. Hua, K., Tao, X., Han, P., Wang, R., and Zhu, J.-K. (2019). Genome Engineering in Rice Using Cas9 Variants that Recognize NG PAM Sequences. Molecular Plant 12,1003-1014.

17. Huang, T. P., Zhao, K. T., Miller, S. M., Gaudelli, N. M., Oakes, B. L., Fellmann, C., Savage, D. F., and Liu, D. R. (2019). Circularly permuted and PAM-modified Cas9 variants broaden the targeting scope of base editors. Nature Biotechnology 37,626-631.

18. Jinek, M., Chylinski, K., Fonfara, I., Hauer, M., Doudna, J. A., and Charpentier, E. (2012). A Programmable Dual-RNA-Guided DNA Endonuclease in Adaptive Bacterial Immunity. Science 337, 816-821.

19. Kearns, N. A., Genga, R. M. J., Enuameh, M. S., Garber, M., Wolfe, S. A., and Maehr, R. (2014). Cas9 effector-mediated regulation of transcription and differentiation in human pluripotent stem cells. Development 141,219-223.

20. Kim, D., Kim, J., Hur, J. K., Been, K. W., Yoon, S., and Kim, J.-S. (2016). Genome-wide analysis reveals specificities of Cpf1 endonucleases in human cells. Nature Biotechnology 34,863-868.

21. Kleinstiver, B. P., Prew, M. S., Tsai, S. Q., Topkar, V. V., Nguyen, N. T., Zheng, Z., Gonzales, A. P. W., Li, Z., Peterson, R. T., Yeh, J.-R.J., et al. (2015). Engineered CRISPR-Cas9 nucleases with altered PAM specificities. Nature 523,481-485.

22. Komor, A. C., Kim, Y. B., Packer, M. S., Zuris, J. A., and Liu, D. R. (2016). Programmable editing of a target base in genomic DNA without double-stranded DNA cleavage. Nature 533,420-424.

23. Meier, J. A., Zhang, F., and Sanjana, N. E. (2017). GUIDES: sgRNA design for loss-of-function screens. Nature Methods 14,831-832.

24. Negishi, K., Kaya, H., Abe, K., Hara, N., Saika, H., and Toki, S. (2019). An adenine base editor with expanded targeting scope using SpCas9-NG v1 in rice. Plant Biotechnology Journal.

25. Nishimasu, H., Shi, X., Ishiguro, S., Gao, L., Hirano, S., Okazaki, S., Noda, T., Abudayyeh, O. O., Gootenberg, J. S., Mori, H., et al. (2018). Engineered CRISPR-Cas9 nuclease with expanded targeting space. Science 361,1259-1262.

26. Ran, F. A., Cong, L., Yan, W. X., Scott, D. A., Gootenberg, J. S., Kriz, A. J., Zetsche, B., Shalem, O., Wu, X., Makarova, K. S., et al. (2015). In vivo genome editing using Staphylococcus aureus Cas9. Nature 520,186-191.

27. Sanjana, N. E., Shalem, O., and Zhang, F. (2014). Improved vectors and genome-wide libraries for CRISPR screening. Nature Methods 11,783-784.

28. Sanson, K. R., Hanna, R. E., Hegde, M., Donovan, K. F., Strand, C., Sullender, M. E., Vaimberg, E. W., Goodale, A., Root, D. E., Piccioni, F., et al. (2018). Optimized libraries for CRISPR-Cas9 genetic screens with multiple modalities. Nature Communications 9.

29. Shalem, O., Sanjana, N. E., Hartenian, E., Shi, X., Scott, D. A., Mikkelsen, T. S., Heckl, D., Ebert, B. L., Root, D. E., Doench, J. G., et al. (2014). Genome-Scale CRISPR-Cas9 Knockout Screening in Human Cells. Science 343, 84-87.

30. Wang, J., Meng, X., Hu, X., Sun, T., Li, J., Wang, K., and Yu, H. (2019). xC as9 expands the scope of genome editing with reduced efficiency in rice. Plant Biotechnology Journal 17, 709-711.

31. Wu, X., Scott, D. A., Kriz, A. J., Chiu, A. C., Hsu, P. D., Dadon, D. B., Cheng, A. W., Trevino, A. E., Konermann, S., Chen, S., et al. (2014). Genome-wide binding of the CRISPR endonuclease Cas9 in mammalian cells. Nature Biotechnology 32, 670-676.

32. Xie, S., Cooley, A., Armendariz, D., Zhou, P., and Hon, G. C. (2018). Frequent sgRNA-barcode recombination in single-cell perturbation assays. PLOS ONE 13, e0198635.

33. Zhang, Y., Ge, X., Yang, F., Zhang, L., Zheng, J., Tan, X., Jin, Z.-B., Qu, J., and Gu, F. (2015). Comparison of non-canonical PAMs for CRISPR/Cas9-mediated DNA cleavage in human cells. Scientific Reports 4.

34. Zhong, Z., Sretenovic, S., Ren, Q., Yang, L., Bao, Y., Qi, C., Yuan, M., He, Y., Liu, S., Liu, X., et al. (2019). Improving Plant Genome Editing with High-Fidelity xCas9 and Non-canonical PAM-Targeting Cas9-NG. Molecular Plant 12, 1027-1036.

What is claimed is:

1. A recombinant Cas9 protein comprising an amino acid sequence that is at least 90% identical to the amino acid sequence of SEQ ID NO: 2, wherein the amino acid sequence of the Cas9 protein comprises:
at least one mutation in an amino acid residue selected from 262, 324, 409, 480, 543, 694, of the amino acid sequence provided in SEQ ID NO: 2 or the corresponding residue of an aligned sequence, and
at least one mutation in an amino acid residue selected from 1111, 1135, 1218, 1219, 1322, 1335, and 1337, of the amino acid sequence provided in SEQ ID NO: 2 or the corresponding residue of an aligned sequence, and wherein the amino acid sequence of the recombinant Cas9 protein is not identical to the amino acid sequence of a naturally occurring Cas9 protein.

2. The recombinant Cas9 protein of claim 1, wherein the mutations are selected from X262T, X324L, X4091, X480K, X543D, X694I, X1111R, X1135V, X1218R, X1219F, X1219V, X1322R, X1335V, and X1337R of the amino acid sequence provided in SEQ ID NO: 2 or the corresponding residue of an aligned sequence, wherein X represents any amino acid.

3. The recombinant Cas9 protein of claim 2, wherein the mutations are selected from A262T, R324L, S409I, E480K, E543D, M694I, L1111R, D1135V, G1218R, E1219F, E1219V, A1322R, R1335V, and T1337R of the amino acid sequence provided in SEQ ID NO: 2.

4. The recombinant Cas9 protein of claim 1, further comprising a mutation in amino acid residue D10, E762, D839, H983, or D986; and at H840 or N863, of the amino acid sequence provided in SEQ ID NO: 2.

5. The recombinant Cas9 protein of claim 5, wherein the mutation is D10A, H840A, or both of the amino acid sequence provided in SEQ ID NO: 2.

6. A recombinant Cas9 protein having the sequence of SEQ ID NO: 1.

7. A fusion protein comprising the recombinant Cas9 protein of claim 1, fused to a heterologous functional domain, with an optional intervening linker, wherein the linker does not interfere with activity of the fusion protein.

8. The fusion protein of claim 7, wherein the heterologous functional domain is a transcriptional activation domain.

9. The fusion protein of claim 7, wherein the heterologous functional domain is a transcriptional silencer or transcriptional repression domain.

10. The fusion protein of claim 7, wherein the heterologous functional domain is an enzyme that modifies the methylation state of DNA.

11. The fusion protein of claim 7, wherein the heterologous functional domain is an enzyme that modifies a histone subunit.

12. The fusion protein of claim 11, wherein the enzyme that modifies a histone subunit is a histone acetyltransferase (HAT), histone deacetylase (HDAC), histone methyltransferase (HMT), or histone demethylase.

13. The fusion protein of claim 7, wherein the heterologous functional domain is a base editor.

14. The fusion protein of claim 7, wherein the heterologous functional domain is a prime editor.

15. The fusion protein of claim 7, wherein the heterologous functional domain is a reverse transcriptase (RT).

16. The fusion protein of claim 7, wherein the heterologous functional domain is a biological tether.

17. The fusion protein of claim 7, wherein the heterologous functional domain comprises a nuclease domain.

18. The fusion protein of claim 7, wherein the heterologous functional domain comprises a recombinase domain.

19. A method of altering the genome of a cell, the method comprising expressing in the cell, or contacting the cell with, the recombinant Cas9 protein of claim 1, and a guide RNA having a region complementary to a selected portion of the genome of the cell.

20. A method of evaluating a CRISPR-Cas system comprising:
a) obtaining a sgRNA library comprising multiple sgRNA sequences which target sites in the genome,
b) cloning said library into a lentiviral plasmid comprising a nucleic acid sequence encoding a Cas protein and, optionally, a barcode,
c) producing lentivirus containing said plasmid,
d) transducing mammalian cells with said lentivirus,
e) culturing said cells for a sufficient time period, and
f) evaluating said cells for CRISPR activity.


SEQUENCE LISTING (56 sequences)

SEQ ID NO: 1 - protein, 1368 aa, Artificial Sequence
  constructed sequence
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 2 - protein, 1368 aa, Streptococcus pyogenes [sequence omitted]

SEQ ID NO: 3 - protein, 1368 aa, Artificial Sequence
  constructed sequence
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 4 - protein, 1368 aa, Artificial Sequence
  Constructed sequence
MDKKYSIGLAIGTNSVGWAVITDEYKVPSKKEKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLIPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVIVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDERKDFQFYKVREINNYHHAHDAYLNAVVGTALIKKYPKLESEFVYGDYKVYDVRKMIAKSEQEIGKATAKYFFYSNIMNFFKTEITLANGEIRKRPLIEINGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, Artificial Sequence
  Constructed sequence
MDKKYSIGLAIGTNSVGWAVITDEYKVPSKKEKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLIPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVIVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDERKDFQFYKVREINNYHHAHDAYLNAVVGTALIKKYPKLESEFVYGDYKVYDVRKMIAKSEQEIGKATAKYFFYSNIMNFFKTEITLANGEIRKRPLIEINGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - DNA, 20 bp, Artificial Sequence
  sgRNA
ccgatcacaaatagtatggg

SEQ ID NO: 7 - DNA, 20 bp, Artificial Sequence
  sgRNA
caaatagtatgggtggcaag

SEQ ID NO: 8 - DNA, 20 bp, Artificial Sequence
  sgRNA
atagtatgggtggcaagagg

SEQ ID NO: 9 - DNA, 20 bp, Artificial Sequence
  sgRNA
tttgtgatcggaatcataca

SEQ ID NO: 10 - DNA, 20 bp, Artificial Sequence
  sgRNA
tccatagcttcaaatgttgg

SEQ ID NO: 11 - DNA, 20 bp, Artificial Sequence
  sgRNA
tcccatttgcagggactgct

SEQ ID NO: 12 - DNA, 20 bp, Artificial Sequence
  sgRNA
gcaaatgggacttacgagtt

SEQ ID NO: 13 - DNA, 20 bp, Artificial Sequence
  sgRNA
aactcgtaagtcccatttgc

SEQ ID NO: 14 - DNA, 20 bp, Artificial Sequence
  sgRNA
ggccaagcagtccctgcaaa

SEQ ID NO: 15 - DNA, 20 bp, Artificial Sequence
  sgRNA
actcgtaagtcccatttgca

SEQ ID NO: 16 - DNA, 20 bp, Artificial Sequence
  sgRNA
gccaagcagtccctgcaaat

SEQ ID NO: 17 - DNA, 20 bp, Artificial Sequence
  sgRNA
cgatcacaaatagtatgggt

SEQ ID NO: 18 - DNA, 20 bp, Artificial Sequence
  sgRNA
aaatagtatgggtggcaaga

SEQ ID NO: 19 - DNA, 20 bp, Artificial Sequence
  sgRNA
tagtatgggtggcaagagga

SEQ ID NO: 20 - DNA, 20 bp, Artificial Sequence
  sgRNA
ttgtgatcggaatcatacat

SEQ ID NO: 21 - DNA, 20 bp, Artificial Sequence
  sgRNA
ccatagcttcaaatgttggt

SEQ ID NO: 22 - DNA, 20 bp, Artificial Sequence
  sgRNA
cccatttgcagggactgctt

SEQ ID NO: 23 - DNA, 20 bp, Artificial Sequence
  sgRNA
caaatgggacttacgagttt

SEQ ID NO: 24 - DNA, 20 bp, Artificial Sequence
  sgRNA
ctaggtgatgatgtcagatt

SEQ ID NO: 25 - DNA, 20 bp, Artificial Sequence
  sgRNA
cagttcatgcagctagcaag

SEQ ID NO: 26 - DNA, 20 bp, Artificial Sequence
  sgRNA
ctgtaagggtcctctttgca

SEQ ID NO: 27 - DNA, 20 bp, Artificial Sequence
  sgRNA
aactgctaggtgatgatgtc

SEQ ID NO: 28 - DNA, 20 bp, Artificial Sequence
  sgRNA
tagctgcatgaactgctagg

SEQ ID NO: 29 - DNA, 20 bp, Artificial Sequence
  sgRNA
cctgcaaagaggacccttac

SEQ ID NO: 30 - DNA, 20 bp, Artificial Sequence
  sgRNA
agttcatgcagctagcaagt

SEQ ID NO: 31 - DNA, 20 bp, Artificial Sequence
  sgRNA
catgcagctagcaagtggtt

SEQ ID NO: 32 - DNA, 20 bp, Artificial Sequence
  sgRNA
aatactgtaagggtcctctt

SEQ ID NO: 33 - DNA, 20 bp, Artificial Sequence
  sgRNA
cgaatctgacatcatcacct

SEQ ID NO: 34 - DNA, 20 bp, Artificial Sequence
  sgRNA
ctaagaacaaaccacttgct

SEQ ID NO: 35 - DNA, 20 bp, Artificial Sequence
  sgRNA
tcctgcaaagaggaccctta

SEQ ID NO: 36 - DNA, 20 bp, Artificial Sequence
  sgRNA
gtaagggtcctctttgcagg

SEQ ID NO: 37 - DNA, 20 bp, Artificial Sequence
  sgRNA
aatctgacatcatcacctag

SEQ ID NO: 38 - DNA, 20 bp, Artificial Sequence
  sgRNA
tgtaagggtcctctttgcag

SEQ ID NO: 39 - DNA, 20 bp, Artificial Sequence
  sgRNA
ggttttactaacttctccaa

SEQ ID NO: 40 - DNA, 20 bp, Artificial Sequence
  sgRNA
ctagcagttcatgcagctag

SEQ ID NO: 41 - DNA, 20 bp, Artificial Sequence
  sgRNA
aaatactgtaagggtcctct

SEQ ID NO: 42 - DNA, 20 bp, Artificial Sequence
  sgRNA
gctgcatgaactgctaggtg

SEQ ID NO: 43 - DNA, 20 bp, Artificial Sequence
  sgRNA
tcatgcagctagcaagtggt

SEQ ID NO: 44 - DNA, 20 bp, Artificial Sequence
  sgRNA
gtaggcgcgccgctctctac

SEQ ID NO: 45 - DNA, 20 bp, Artificial Sequence
  sgRNA
tactaacgccgctcctacag

SEQ ID NO: 46 - DNA, 20 bp, Artificial Sequence
  sgRNA
acggaggctaagcgtcgcaa

SEQ ID NO: 47 - DNA, 24 bp, Artificial Sequence
  primer
tcttgtggaaaggacgaaacaccg

SEQ ID NO: 48 - DNA, 23 bp, Artificial Sequence
  primer
ccgactcggtgccactttttcaa

SEQ ID NO: 49 - DNA, 99 bp, Artificial Sequence
  primer
aatgatacggcgaccaccgagatctacactctttccctacacgacgctcttccgatctacgatcgataggtaaggtcttgtggaaaggacgaaacaccg

SEQ ID NO: 50 - DNA, 98 bp, Artificial Sequence
  primer
caagcagaagacggcatacgagataggtaagggtgactggagttcagacgtgtgctcttccgatctacgatcgatccgactcggtgccactttttcaa

SEQ ID NO: 51 - DNA, 44 bp, Artificial Sequence
  primer
tcttgtggaaaggacgaaacaccgtacctgctgccagaccacag

SEQ ID NO: 52 - DNA, 43 bp, Artificial Sequence
  primer
ccgactcggtgccactttttcaagtatgggtggcaagaggagg

SEQ ID NO: 53 - DNA, 44 bp, Artificial Sequence
  primer
tcttgtggaaaggacgaaacaccggaagctatggagctcattgg

SEQ ID NO: 54 - DNA, 50 bp, Artificial Sequence
  primer
ccgactcggtgccactttttcaaaagaggtttgtttacttactataacag

SEQ ID NO: 55 - DNA, 44 bp, Artificial Sequence
  primer
tcttgtggaaaggacgaaacaccgctttcaggagaaacatgtcc

SEQ ID NO: 56 - DNA, 46 bp, Artificial Sequence
  primer
ccgactcggtgccactttttcaattactaacacttcccttatttcc